(19) United States
(12) Patent Application Publication
(10) Pub. No.: US 2024/0050567 A1
(43) Pub. Date: Feb. 15, 2024

(54) MODIFIED IMMUNE EFFECTOR CELL AND USE THEREOF

(71) Applicant: SHANGHAI JIAO TONG UNIVERSITY, Shanghai (CN)

(72) Inventors: Xuanming YANG, Shanghai (CN); Haiyong WANG, Shanghai (CN)

(73) Assignee: SHANGHAI JIAO TONG UNIVERSITY, Shanghai (CN)

(21) Appl. No.: 18/260,562

(22) PCT Filed: Jan. 4, 2022
(86) PCT No.: PCT/CN2022/070012

§ 371 Date: Jul. 6, 2023

(30) Foreign Application Priority Data
Jan. 7, 2021 (CN) 202110017842.8

(51) Int. Cl. A61K 39/00; C07K 14/725; C07K 14/47; C07K 16/28; C07K 14/705; C07K 14/735; C07K 14/715; A61P 35/00

(52) U.S. Cl. CPC A61K 39/4631; C07K 14/7051; C07K 14/4747; C07K 16/2887; C07K 16/28; C07K 14/70521; C07K 14/70517; C07K 14/70596; C07K 14/70535; C07K 14/7056; C07K 14/70589; C07K 14/70532; C07K 14/7151; C07K 14/70575; A61P 35/00; C07K 2317/622; C07K 2319/03

(57) ABSTRACT

An immune effector cell, including and/or expressing a chimeric antigen receptor (CAR), and a Bcl-2 protein or a functionally active fragment thereof. A composition including the immune effector cell. A method for treating diseases and/or disorders, including administering to a subject in need thereof the immune effector cell, where the diseases and/or disorders include tumors.

### FIELD OF THE INVENTION

The present application relates to the field of biomedicine, and specifically to a modified CAR-T cell and a use thereof.

### BACKGROUND OF THE INVENTION

CLDN18 is a member of the Claudins protein family, and CLDN18.1 and CLDN18.2 are alternative splicing forms of CLDN18. In normal tissues, CLDN18.1 is mainly expressed in lung, and CLDN18.2 is only expressed on gastric mucosal epithelium cells. However, CLDN18.2 is expressed in a variety of tumor tissues, such as gastric cancer, pancreatic cancer, esophagus cancer, ovarian cancer, lung cancer, etc., and is an ideal target for tumor CAR-T therapy.

CD20 is specifically expressed in B cells or leukemia or lymphoma derived from B cells. MabThera, an antibody drug targeting CD20, has been marketed, but there are drug resistance and recurrence during the treatment.

Glypican-3 (GPC-3) is not expressed in normal liver tissues but is highly expressed in fetal liver and liver cancer tissues. It is a specific marker for primary hepatocellular carcinoma and is involved in the development and progression of liver cancer, therefore, it is a target for the small molecule targeted therapy of liver cancer, as well as a recognition target for immunotherapy.

In adoptive cell therapy, chimeric antigen receptor T cells (CAR-T cells) are artificially modified tumor killing cells, which combine the target recognition function of antibodies and the tumor killing function of T cells and thus become a major breakthrough in the field of tumor immunotherapy. However, the efficacy of CAR-T on the treatment of solid tumors such as gastric cancer and pancreatic cancer is not satisfactory, and novel CAR-T therapies are the key to the treatment of solid tumors.

### SUMMARY OF THE INVENTION

The present application provides an immune effector cell, which includes and/or expresses a chimeric antigen receptor (CAR), and a Bcl-2 protein or a functionally active fragment thereof. The immune effector cell has one or more of the following properties: 1) capable of specifically binding to an antigen; 2) having a strong in vitro expansion ability; 3) having a strong in vivo anti-tumor ability; 4) having a strong in vitro anti-tumor ability.

In one aspect, the present application provides an immune effector cell, which includes and/or expresses a chimeric antigen receptor (CAR) and a Bcl-2 protein or a functionally active fragment thereof.

In some embodiments, the CAR includes an antigen binding domain, and the antigen binding domain includes an antibody specifically binding to CD20 or an antigen binding fragment thereof.

In some embodiments, the antibody or the antigen binding fragment thereof includes a HCDR3, and the HCDR3 includes an amino acid sequence as set forth in SEQ ID NO: 1.

In some embodiments, the antibody or the antigen binding fragment thereof includes a HCDR2, and the HCDR2 includes an amino acid sequence as set forth in SEQ ID NO: 2.

In some embodiments, the antibody or the antigen binding fragment thereof includes a HCDR1, and the HCDR1 includes an amino acid sequence as set forth in SEQ ID NO: 3.

In some embodiments, the antibody or the antigen binding fragment thereof includes a LCDR3, and the LCDR3 includes an amino acid sequence as set forth in SEQ ID NO: 4.

In some embodiments, the antibody or the antigen binding fragment thereof includes a LCDR2, and the LCDR2 includes an amino acid sequence as set forth in SEQ ID NO: 5.

In some embodiments, the antibody or the antigen binding fragment thereof includes a LCDR1, and the LCDR1 includes an amino acid sequence as set forth in SEQ ID NO: 6.

In some embodiments, the antibody or the antigen binding fragment thereof includes a VH, and the VH includes an amino acid sequence as set forth in SEQ ID NO: 7.

In some embodiments, the antibody or the antigen binding fragment thereof includes a VL, and the VL includes an amino acid sequence as set forth in SEQ ID NO: 8.

In some embodiments, the antibody or the antigen binding fragment thereof includes an amino acid sequence as set forth in SEQ ID NO: 10.

In some embodiments, the CAR includes an antigen binding domain, and the antigen binding domain includes an antibody specifically binding to CLDN18.2 or an antigen binding fragment thereof.

In some embodiments, the antibody or the antigen binding fragment thereof includes a HCDR3, and the HCDR3 includes an amino acid sequence as set forth in SEQ ID NO: 11.

In some embodiments, the antibody or the antigen binding fragment thereof includes a HCDR2, and the HCDR2 includes an amino acid sequence as set forth in SEQ ID NO: 12.

In some embodiments, the antibody or the antigen binding fragment thereof includes a HCDR1, and the HCDR1 includes an amino acid sequence as set forth in SEQ ID NO: 13.

In some embodiments, the antibody or the antigen binding fragment thereof includes a LCDR3, and the LCDR3 includes an amino acid sequence as set forth in SEQ ID NO: 14.

In some embodiments, the antibody or the antigen binding fragment thereof includes a LCDR2, and the LCDR2 includes an amino acid sequence as set forth in SEQ ID NO: 15.

In some embodiments, the antibody or the antigen binding fragment thereof includes a LCDR1, and the LCDR1 includes an amino acid sequence as set forth in SEQ ID NO: 16.

In some embodiments, the antibody or the antigen binding fragment thereof includes a VH, and the VH includes an amino acid sequence as set forth in SEQ ID NO: 17.

In some embodiments, the antibody or the antigen binding fragment thereof includes a VL, and the VL includes an amino acid sequence as set forth in SEQ ID NO: 18.

In some embodiments, the antibody or the antigen binding fragment thereof includes an amino acid sequence as set forth in SEQ ID NO: 19.

In some embodiments, the CAR includes an antigen binding domain, and the antigen binding domain includes an antibody specifically binding to GPC-3 or an antigen binding fragment thereof.

In some embodiments, the antibody or the antigen binding fragment thereof includes a HCDR3, and the HCDR3 includes an amino acid sequence as set forth in any one of SEQ ID NO: 20, SEQ ID NO: 29, and SEQ ID NO: 38.

In some embodiments, the antibody or the antigen binding fragment thereof includes a HCDR2, and the HCDR2 includes an amino acid sequence as set forth in any one of SEQ ID NO: 21, SEQ ID NO: 30, and SEQ ID NO: 39.

In some embodiments, the antibody or the antigen binding fragment thereof includes a HCDR1, and the HCDR1 includes an amino acid sequence as set forth in any one of SEQ ID NO: 22, SEQ ID NO: 31, and SEQ ID NO: 40.

In some embodiments, the antibody or the antigen binding fragment thereof includes a LCDR3, and the LCDR3 includes an amino acid sequence as set forth in any one of SEQ ID NO: 23, SEQ ID NO: 32, and SEQ ID NO: 41.

In some embodiments, the antibody or the antigen binding fragment thereof includes a LCDR2, and the LCDR2 includes an amino acid sequence as set forth in any one of SEQ ID NO: 24, SEQ ID NO: 33, and SEQ ID NO: 42.

In some embodiments, the antibody or the antigen binding fragment thereof includes a LCDR1, and the LCDR1 includes an amino acid sequence as set forth in any one of SEQ ID NO: 25, SEQ ID NO: 34, and SEQ ID NO: 43.

In some embodiments, the antibody or the antigen binding fragment thereof includes a VH, and the VH includes an amino acid sequence as set forth in any one of SEQ ID NO: 26, SEQ ID NO: 35, and SEQ ID NO: 44.

In some embodiments, the antibody or the antigen binding fragment thereof includes a VL, and the VL includes an amino acid sequence as set forth in any one of SEQ ID NO: 27, SEQ ID NO: 36, and SEQ ID NO: 45.

In some embodiments, the antibody or the antigen binding fragment thereof includes an amino acid sequence as set forth in any one of SEQ ID NO: 28, SEQ ID NO: 37, and SEQ ID NO: 46.

In some embodiments, the antibody includes a single-chain antibody.

In some embodiments, the CAR includes a transmembrane domain, and the transmembrane domain includes a transmembrane domain derived from a protein selected from the group consisting of: CD28, CD3e, CD45, CD5, CD8, CD9, CD16, CD22, CD33, CD37, CD64, CD80, CD86, CD134, CD137, and CD154.

In some embodiments, the transmembrane domain includes an amino acid sequence as set forth in SEQ ID NO: 47.

In some embodiments, the CAR includes a co-stimulatory domain, and the co-stimulatory domain includes one or more co-stimulatory domains of a protein selected from the group consisting of: co-stimulatory signaling regions in CD28, 4-1BB, CD40L, TIM1, CD226, DR3, SLAM, ICOS, OX40, NKG2D, 2B4, CD244, FcϵRIγ, BTLA, CD27, CD30, GITR, HVEM, DAP10, CD2, NKG2C, LIGHT, and DAP12.

In some embodiments, the co-stimulatory domain includes an amino acid sequence as set forth in SEQ ID NO: 48.

In some embodiments, the CAR includes an intracellular signaling domain, and the intracellular signaling domain includes an intracellular signaling domain derived from CD3.

In some embodiments, the intracellular signaling domain includes an amino acid sequence as set forth in SEQ ID NO: 49.

In some embodiments, the CAR includes a hinge region, and the hinge region is located between the antigen binding domain and the transmembrane domain.

In some embodiments, the hinge region includes at least one of hinge regions of CD8, CD28, 4-1BB, CD4, CD27, CD7, and PD-1.

In some embodiments, the hinge region includes an amino acid sequence as set forth in SEQ ID NO: 50.

In some embodiments, the CAR includes an amino acid sequence as set forth in any one of SEQ ID NO: 53, SEQ ID NO: 55, SEQ ID NO: 57, SEQ ID NO: 59, and SEQ ID NO: 61.

In some embodiments, the Bcl-2 protein or the functionally active fragment thereof includes an exogenous Bcl-2 protein or a functionally active fragment thereof.

In some embodiments, the Bcl-2 protein or the functionally active fragment thereof includes an amino acid sequence as set forth in SEQ ID NO: 52.

In some embodiments, the immune effector cell includes T cells.

In another aspect, the present application further provides a nucleic acid molecule encoding the CAR and the Bcl-2 protein or the functionally active fragment thereof.

In some embodiments, the nucleic acid molecule includes a sequence encoding a self-cleaving peptide located between a sequence encoding the CAR and a sequence encoding the Bcl-2 protein.

In some embodiments, the self-cleaving peptide includes a 2A peptide.

In some embodiments, the 2A peptide is one or more selected from the group consisting of: P2A, T2A, E2A, and F2A.

In some embodiments, the 2A peptide includes an amino acid sequence as set forth in SEQ ID NO: 51.

In another aspect, the present application further provides a vector including the nucleic acid molecule.

In some embodiments, the vector is selected from one or more of plasmids, retroviral vectors, and lentiviral vectors.

In another aspect, the present application further provides an immune effector cell including the nucleic acid molecule or the vector.

In another aspect, the present application further provides a method for preparing the immune effector cell, which includes culturing the immune effector cell under conditions allowing the expression of the chimeric antigen receptor.

In another aspect, the present application further provides a composition including the immune effector cell.

In another aspect, the present application further provides use of the immune effector cell, the nucleic acid molecule, the vector, or the composition in the preparation of drugs for preventing and/or treating diseases and/or disorders.

In another aspect, the present application further provides the immune effector cell, the nucleic acid molecule, the vector, or the composition, which is used for preventing and/or treating diseases and/or disorders.

In another aspect, the present application further provides a method for preventing and/or treating diseases and/or disorders, and the method includes administering to a subject in need the immune effector cell, the nucleic acid molecule, the vector, or the composition of the present application.

In some embodiments, the diseases and/or disorders are associated with the expression of CD20.

In some embodiments, the diseases and/or disorders are associated with the expression of CLDN18.2.

In some embodiments, the diseases and/or disorders are associated with the expression of GPC-3.

In some embodiments, the diseases and/or disorders include tumors.

In some embodiments, the tumors include solid tumors and/or blood tumors.

In some embodiments, the tumors include CD20 positive tumors.

In some embodiments, the tumors include CLDN18.2 positive tumors.

In some embodiments, the tumors include GPC-3 positive tumors.

In some embodiments, the tumors include lymphoma.

In some embodiments, the tumors include pancreatic cancer.

Those skilled in the art can easily perceive other aspects and advantages of the present application from the detailed description below. In the following detailed description, only exemplary embodiments of the present application are shown and described. As those skilled in the art will recognize, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWINGS

The specific features of the invention involved in the present application are shown in the appended claims. The characteristics and advantages of the invention involved in the present application can be better understood by referring to the exemplary embodiments and the accompanying drawings described in detail below. A brief description of the drawings is as follows:

FIG. 1 shows a schematic diagram of the construction of the chimeric antigen receptor of the present application.

FIGS. 2A-2B show the expression profile of the CAR of the present application.

FIG. 3 shows the expression profile of the CAR of the immune effector cell of the present application.

FIG. 4 shows the expansion capacity of anti-CD20 Bcl-2 CAR-T cells.

FIG. 5 shows the expansion capacity of anti-CLDN18.2 Bcl-2 CAR-T cells.

FIG. 6 shows the expansion capacity of anti-GPC-3 Bcl-2 CAR-T cells.

FIG. 7 shows the in vitro tumor killing capacity of the immune effector cell of the present application.

FIGS. 8A-8B show the in vivo tumor killing capacity of the immune effector cell of the present application.

FIG. 9 shows the effect of anti-CLDN18.2 Bcl-2 CAR-T cells on tumor size.

FIG. 10 shows the in vivo tumor killing capacity of anti-GPC-3 Bcl-2 CAR-T cells.

### DETAILED DESCRIPTION OF THE EMBODIMENTS

The implementation of the present application will be illustrated in the following specific examples, and other advantages and effects of the present application will be easily known by those familiar with this technology from the content disclosed in the specification.

### DEFINITION OF TERMS

In the present application, the term “immune effector cell” generally refers to an immune cell that is involved in the immune response and performs an effector function. For example, the exercise of an effector function may include removal of foreign antigens or promotion of immune effector responses. The immune effector cells may include plasmocytes, T cells, B cells, natural killer (NK) cells, natural killer T (NKT) cells, mastocytes, and bone marrow-derived phagocytes. The immune effector cell of the present application may include engineered immune effector cells. For example, the immune effector cell of the present application may include a chimeric antigen receptor and may also express a Bcl-2 protein or a functionally active fragment thereof. For example, the Bcl-2 protein is an exogenous Bcl-2 protein or a functionally active fragment thereof.

In the present application, the term “chimeric antigen receptor” or “CAR”, also referred to as “chimeric receptor”, “T receptor”, or chimeric immune receptor, generally refers to a recombinant polypeptide construct including at least a extracellular antigen binding domain, a transmembrane domain and a cytoplasmic signaling domain (also referred to as “intracellular signaling domain”). For example, the chimeric antigen receptor may include a targeting moiety (e.g., the moiety binding to a tumor-related antigen), a hinge region, a transmembrane domain, a co-stimulatory domain, and an intracellular signaling domain.

The terms “Bcl-2” and “Bcl-2 protein” may be used interchangeably and generally refer to the encoded products of bcl-2 proto-oncogenes. In some embodiments, the amino acid sequence of Bcl-2 protein may be as set forth in SEQ ID NO: 52. In the present application, the term covers full-length Bcl-2 proteins and homologues, analogues, truncations, mutants, and functionally active fragments thereof. In the present application, the term covers exogenous Bcl-2 proteins or functionally active fragments thereof.

In the present application, the term “antigen binding domain” generally refers to a domain capable of binding to a target antigen. The antigen binding domain may include a chimeric antigen receptor, a fragment thereof, an antibody or an antigen binding fragment thereof, which is capable of (specifically) binding to an antigen. The antigen binding domain may be of natural, synthetic, semi-synthetic or recombinant origin. In some embodiments, the antigen binding domain may include an antibody or an antigen binding fragment thereof. For example, the antigen binding domain may include a single-chain antibody.

In the present application, the term “specifically binding to” or “specific” generally refers to measurable and reproducible interactions, e.g., the binding between a target and an antibody, and may determine the presence of a target in the presence of a heterogeneous population of molecules (including biomolecules). For example, the antibody that specifically binds a target (that may be an epitope) may be an antibody that binds the target with greater affinity, avidity, easier, and/or for a longer duration than it binds other targets. In some embodiments, the antibody specifically binds an epitope on a protein, the epitope being conserved in different species of proteins. In some embodiments, specific binding may include but does not require exclusive binding.

In the present application, the term “antibody” generally refers to a polypeptide molecule capable of specifically recognizing and/or neutralizing a particular antigen. For example, the antibody may include an immunoglobulin composed of at least two heavy (H) chains and two light (L) chains linked to each other through disulfide bonds, and includes any molecules containing the antigen binding moiety. The term “antibody” includes monoclonal antibodies, antibody fragments or antibody derivatives, including, but not limited to, human antibodies, humanized antibodies, chimeric antibodies, single-domain antibodies (e.g., dAb), single-chain antibodies (e.g., scFv), and antibody fragments binding to antigens (e.g., Fab, Fab′ and (Fab)₂ fragments). The term “antibody” also includes all recombinant forms of the antibody, for example, antibodies expressed in prokaryotic cells, unglycosylated antibodies, as well as any antibody fragments binding to antigens of the present application and derivatives thereof. Each heavy chain may be composed of a heavy chain variable region (VH) and a heavy chain constant region. Each light chain may be composed of a light chain variable region (VL) and a light chain constant region. VH and VL regions may be further distinguished into highly variable regions called complementary determining regions (CDRs), which are interspersed in more conserved regions called framework regions (FRs). Each of VH and VL may be composed of three CDRs and four FRs, which can be arranged in the following order from the amino terminus to the carboxyl terminus: FR1, CDR1, FR2, CDR2, FR3, CDR3, and FR4. The heavy chain and light chain variable regions contain binding domains interacting with antigens.

In the present application, the term “CDR”, also referred to as “complementary determining region”, generally refers to a region in the variable domain of an antibody, the sequence of which is highly variable and/or forms the structure-defining loop. Generally, an antibody includes six CDRs; three in VH (HCDR1, HCDR2, HCDR3), and three in VL (LCDR1, LCDR2, LCDR3). In some embodiments, naturally occurring camelid antibodies composed only of heavy chains may also function normally and stably in the absence of light chains. See, e.g., Hamers-Casterman et al., Nature 363: 446-448 (1993); Sheriff et al, Nature Struct. Biol. 3:733-736 (1996). The CDRs of an antibody may be determined by various coding systems, such as CCG, Kabat, Chothia, IMGT, comprehensive consideration of Kabat/Chothia, etc. These coding systems are known in the art, specifically see, e.g., http://www.bioinf.org.uk/abs/index.html #kabatnum. For example, the amino acid sequence number of the antibody may follow the IMGT numbering scheme (IMGT, the international ImMunoGeneTics information system@imgt.cines.fr; http://imgt.cines.fr; Lefranc et, al., 1999, Nucleic Acids Res. 27: 209-212; Ruiz et, al., 2000 Nucleic Acids Res. 28: 219-221; Lefranc et, al., 2001, Nucleic Acids Res. 29:207-209; Lefranc et, al., 2003, Nucleic Acids Res. 31: 307-310; Lefranc et, al., 2005, DevComp Immunol 29: 185-203). For example, the CDRs of the antibody may be determined by the Kabat numbering system (see e.g., Kabat E A &Wu T T (1971) Ann NY AcadSci 190:382-391 and Kabat E A et al., (1991) Sequences of Proteins of Immunological Interest, Fifth Edition, U.S. Department of Health and Human Services, NIH Publication No. 91-3242).

In the present application, the term “FR” generally refers to the more highly conserved portions of the variable domain of an antibody, which are referred to as framework regions. Generally, the variable domains of natural heavy chains and light chains each include four FRs, i.e., four in VH (H-FR1, H-FR2, H-FR3, and H-FR4), and four in VL (L-FR1, L-FR2, L-FR3, and L-FR4).

In the present application, the terms “variable domain” and “variable region” may be used interchangeably and generally refer to a portion of the heavy chain and/or light chain of an antibody. The variable domains of heavy chains and light chains may be respectively referred to as “VII” and “V_{L}” (or referred to as “VH” and “VL”, respectively). These domains are usually the most variable part of an antibody (relative to other antibodies of the same type) and include antigen binding sites.

In the present application, the term “single-chain antibody (scFv)” generally refers to a fusion protein including at least one antibody fragment containing a light chain variable region and at least one antibody fragment containing a heavy chain variable region. Where, the light chain variable region and the heavy chain variable region are contiguous (e.g., via a synthetic linker, such as a short flexible polypeptide linker). The scFv may be expressed as a single-chain polypeptide, and may also retain the specificity of the intact antibody from which it is derived. In the present application, the scFv may contain the VL and VH in any order (e.g., relative to the N terminus and C terminus of the polypeptide), and the scFv may also include VL-linker-VH or VH-linker-VL.

In the present application, the term “transmembrane domain” generally refers to a domain capable of spanning the cytoplasmic membrane. The transmembrane domain may usually contain three structural regions: an N-terminal extracellular region, an intermediate transmembrane extension region, and a C-terminal cytoplasmic region. The transmembrane domain may also contain an intracellular region or a cytoplasmic region.

In the present application, the term “co-stimulatory domain” generally refers to an intracellular domain that can provide immune co-stimulatory molecules. Where, the co-stimulatory molecules may be cell surface molecule required for an effective response of the lymphocyte to the antigen. In some embodiments, the co-stimulatory domain may be the intracellular part of the co-stimulatory molecules or the truncated forms thereof.

In the present application, the term “intracellular signaling domain” generally refers to a domain located inside a cell capable of conducting signals. In the present application, the intracellular signaling domain can conduct signals into the cells. The term covers intracellular signaling domains capable of inducing effector function signals and any truncated portions thereof.

In the present application, the term “4-1BB”, also referred to as “CD137”, generally refers to a member of the tumor necrosis factor (TNF) receptor family and is encoded by the tumor necrosis factor receptor superfamily member 9 (TNFRSF9) gene. The amino acid sequence of human 4-1BB can be found in GenBank Accession Number No. AAA62478.2. The 4-1BB of the present application covers the homologues, analogues, or mutants thereof.

In the present application, the “vector” generally refers to a nucleic acid molecule capable of self-replication in a suitable host, which is used to transfer the inserted nucleic acid molecule into and/or between host cells. The vector may include a vector mainly used for inserting DNA or RNA into cells, a vector mainly used for replicating DNA or RNA, and a vector mainly used for expression of DNA or RNA transcription and/or translation. The vector also includes a vector with a variety of the above functions. The vector may be a polynucleotide that can be transcribed and translated into a polypeptide when introduced into a suitable host cell. Generally, by culturing a suitable host cell containing the vector, the vector can produce the desired expression products.

In the present application, the term “plasmid” generally refers to DNA molecules other than chromosomes or nucleoids in organisms such as bacteria and yeast, which is present in the cytoplasm and have the ability to replicate autonomously, enabling them to maintain a constant copy number in offspring cells and express the genetic information carried. In genetic engineering research, plasmids are used as gene vectors.

In the present application, the term “retroviral vector” generally refers to viral particles that can control and express exogenous genes but cannot self-package into proliferative virus particles. Such viruses mostly contain reverse transcriptase. Retroviruses contain at least three genes: gag which contains the gene for the protein that makes up the center and structure of the virus; pol which contains the gene for the reverse transcriptase; and env which contains the gene that makes up the outer shell of the virus. Through retroviral transfection, retroviral vectors can randomly and stably integrate their own genome and the exogenous genes they carry into the host cell genome, for example, they can integrate CAR molecules into the host cell.

In the present application, the term “lentiviral vector” generally refers to a diploid RNA viral vector that is a retrovirus. Lentiviral vectors are vectors prepared by, based on the genome of the lentivirus, removing multiple sequence structures related to the viral activity to make it biologically safe, and then introducing the sequence and expression structure of the target gene required for the experiment into the genome backbone. Through lentiviral vector transfection, retroviral vectors can randomly and stably integrate their own genome and the exogenous genes they carry into the host cell genome, for example, they can integrate CAR molecules into the host cell.

The term “tumor” generally refers to any new pathological tissue hyperplasia. Tumor cells may spread to other parts of the body locally or through the bloodstream and lymphatic system. In the present application, the tumors may include benign tumors and malignant tumors. In the present application, the tumors may include solid tumors and/or blood tumors. In the present application, the tumors may include cancers. In the present application, examples of tumors include, but not limited to brain glioma, breast cancer, melanoma, non-small cell lung cancer, bladder cancer, ovarian cancer, and colorectal cancer.

In the present application, it should also be understood that the involved protein, polypeptide and/or amino acid sequences includes at least the following scope: variants or homologues having the same or similar functions as the protein or polypeptide.

In the present application, the variants may be proteins or polypeptides with one or more amino acid substitutions, deletions, or additions in the amino acid sequence of the protein and/or the polypeptide (e.g., antibodies or fragments thereof that specifically bind to CD73 protein). For example, the functional variants may include proteins or polypeptides with amino acid changes by at least 1, for example, 1-30, 1-20 or 1-10, and further for example 1, 2, 3, 4 or 5 amino acid substitutions, deletions and/or insertions. The functional variants may substantially retain the biological properties of the protein or the polypeptide before change (e.g., substitution, deletion, or addition). For example, the functional variants may retain at least 60%, 70%, 80%, 90%, or 100% of the biological activity (e.g., antigen-binding ability) of the protein or the polypeptide before change. For example, the substitution may be conservative substitution.

In the present application, the homologues may be proteins or polypeptides having at least about 85% (e.g., having at least about 85%, about 90%, about 91%, about 92%, about 93%, about 94%, about 95%, about 96%, about 97%, about 98%, about 99% or higher) sequence homology with the amino acid sequence of the protein and/or the polypeptide (e.g., antibodies or fragments thereof that specifically bind to CD73 protein).

In the present application, the homology generally refers to similarity, analogy or association between two or more sequences. The “percentage of sequence homology” can be calculated as below: comparing two sequences to be compared in a comparison window, determining the number of positions where the same nucleic acid bases (e.g., A, T, C, G, I) or the same amino acid residues (e.g., Ala, Pro, Ser, Thr, Gly, Val, Leu, Ile, Phe, Tyr, Trp, Lys, Arg, His, Asp, Glu, Asn, Gln, Cys, and Met) are present in both sequences so as to obtain the number of matching positions, dividing the number of matching positions by the total number of positions in the comparison window (i.e., window size), multifying the result by 100 to generate the percentage of sequence homology. The alignment for determining the percentage of sequence homology can be achieved in a variety of ways known in the art, for example, by using publicly available computer software, such as BLAST, BLAST-2. ALIGN, or Megalign (DNASTAR) software. A person skilled in the art can determine suitable parameters for aligning sequences, including any algorithms needed to achieve the maximal alignment over the full-length sequence range being compared or within the target sequence region. The homology can also be determined by the following methods: FASTA and BLAST. The description of FASTA algorithm can be found in “Improved tools for biological sequence comparison” to W. R. Pearson and D. J. Lipman. Proc. Nat. Acad. Sci., 85: 2444-2448, 1988; and “Rapid and Sensitive Protein Similarity Searches” to D. J. Lipman and W. R. Pearson, Science, 227: 1435-1441, 1989. The description of BLAST algorithm can be found in “Basic Local Alignment Search Tool” to S. Altschul, W. Gish, W Miller. E. W, Myers, and D. Lipman. Journal of Molecular Biology, 215:403-410, 1990.

In the present application, the term “include” generally refers to the meaning of comprising, encompassing, containing, or embracing. In some cases, it also means “is/are” or “be composed of”.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 65%, 7%, 7.5%, 8%, 8.5%, 9%, 9.5%, or 10% above or below a specified value.

### DETAILED DESCRIPTION OF THE INVENTION

Immune Effector Cell

In one aspect, the present application provides an immune effector cell, which may include and/or express a chimeric antigen receptor (CAR), and the immune effector cell may further include and/or express a Bcl-2 protein or a functionally active fragment thereof. The Bcl-2 protein or the functionally active fragment thereof may be introduced into the immune effector cell exogenously (e.g., artificially synthesizing the following sequences: a hinge region, a transmembrane domain, a co-stimulatory domain, an intracellular signaling domain, a cleavage peptide, and a Bcl-2 or a functionally active fragment thereof, linking the various parts and then adding scFv, molecule cloning to produce viral vectors, and infecting the immune effector cell for expression). For example, the immune effector cell (e.g., T cells) may be an engineered or modified immune effector cell (e.g., T cells), wherein the engineering may include introducing into the immune effector cell (e.g., immune effector cells from other natural sources derived from subjects) the CAR and/or the Bcl-2 protein, and/or nucleic acid molecules expressing the CAR and/or the Bcl-2 protein (e.g., by constructing nucleic acid sequences encoding the antigen binding protein, hinge region, transmembrane domain, co-stimulatory domain, intracellular signaling domain, cleavage peptide, and Bcl-2, and infecting the immune effector cell with the viral vector for expression). In some embodiments, the CAR includes an antigen binding domain, a transmembrane domain, a co-stimulatory domain, an intracellular signaling domain, and/or a hinge region. In some embodiments, the immune effector cell over-expresses the Bcl-2 protein or the functionally active fragment thereof.

In some embodiments, the CAR includes an antigen binding domain, and the antigen binding domain includes an antibody targeting CD20 or an antigen binding fragment thereof.

In some embodiments, the antibody targeting CD20 or the antigen binding fragment thereof includes a HCDR3, and the HCDR3 includes an amino acid sequence as set forth in SEQ ID NO: 1; in some embodiments, the antibody or the antigen binding fragment thereof includes a HCDR2, and the HCDR2 includes an amino acid sequence as set forth in SEQ ID NO: 2; in some embodiments, the antibody or the antigen binding fragment thereof includes a HCDR1, and the HCDR1 includes an amino acid sequence as set forth in SEQ ID NO: 3. In some embodiments, the antibody or the antigen binding fragment thereof includes a LCDR3, and the LCDR3 includes an amino acid sequence as set forth in SEQ ID NO: 4; in some embodiments, the antibody or the antigen binding fragment thereof includes a LCDR2, and the LCDR2 includes an amino acid sequence as set forth in SEQ ID NO: 5; in some embodiments, the antibody or the antigen binding fragment thereof includes a LCDR1, and the LCDR1 includes an amino acid sequence as set forth in SEQ ID NO: 6.

In some embodiments, the antibody or the antigen binding fragment thereof includes a HCDR3, a HCDR2, and a HCDR1, the HCDR3 includes an amino acid sequence as set forth in SEQ ID NO: 1, the HCDR2 includes an amino acid sequence as set forth in SEQ ID NO: 2, and the HCDR1 includes an amino acid sequence as set forth in SEQ ID NO: 3.

In some embodiments, the antibody or the antigen binding fragment thereof includes a LCDR3, a LCDR2, and a LCDR1, the LCDR3 includes an amino acid sequence as set forth in SEQ ID NO: 4, the LCDR2 includes an amino acid sequence as set forth in SEQ ID NO: 5, and the LCDR1 includes an amino acid sequence as set forth in SEQ ID NO: 6.

In some embodiments, the antibody or the antigen binding fragment thereof includes a HCDR3, a HCDR2, a HCDR1, a LCDR3, a LCDR2, and a LCDR1, the HCDR3 includes an amino acid sequence as set forth in SEQ ID NO: 1, the HCDR2 includes an amino acid sequence as set forth in SEQ ID NO: 2, the HCDR1 includes an amino acid sequence as set forth in SEQ ID NO: 3, the LCDR3 includes an amino acid sequence as set forth in SEQ ID NO: 4, the LCDR2 includes an amino acid sequence as set forth in SEQ ID NO: 5, and the LCDR1 includes an amino acid sequence as set forth in SEQ ID NO: 6.

In some embodiments, the antibody or the antigen binding fragment thereof includes a VH, and the VH may include an amino acid sequence as set forth in SEQ ID NO: 7. In some embodiments, the antibody or the antigen binding fragment thereof includes a VL, and the VL may include an amino acid sequence as set forth in SEQ ID NO: 8.

In some embodiments, the antibody or the antigen binding fragment thereof includes a VH and a VL, the VH includes an amino acid sequence as set forth in SEQ ID NO: 7 and the VL includes an amino acid sequence as set forth in SEQ ID NO: 8.

In some embodiments, the antibody or the antigen binding fragment thereof includes an amino acid sequence as set forth in SEQ ID NO: 10.

In some embodiments, the CAR includes an antigen binding domain, and the antigen binding domain includes an antibody targeting CLDN18.2 or an antigen binding fragment thereof.

In some embodiments, the antibody targeting CLDN18.2 or the antigen binding fragment thereof includes a HCDR3, and the HCDR3 includes an amino acid sequence as set forth in SEQ ID NO: 11; in some embodiments, the antibody or the antigen binding fragment thereof includes a HCDR2, and the HCDR2 includes an amino acid sequence as set forth in SEQ ID NO: 12; in some embodiments, the antibody or the antigen binding fragment thereof includes a HCDR1, and the HCDR1 includes an amino acid sequence as set forth in SEQ ID NO: 13. In some embodiments, the antibody or the antigen binding fragment thereof includes a LCDR3, and the LCDR3 includes an amino acid sequence as set forth in SEQ ID NO: 14; in some embodiments, the antibody or the antigen binding fragment thereof includes a LCDR2, and the LCDR2 includes an amino acid sequence as set forth in SEQ ID NO: 15; in some embodiments, the antibody or the antigen binding fragment thereof includes a LCDR1, and the LCDR1 includes an amino acid sequence as set forth in SEQ ID NO: 16.

In some embodiments, the antibody or the antigen binding fragment thereof includes a HCDR3, a HCDR2, and a HCDR1, the HCDR3 includes an amino acid sequence as set forth in SEQ ID NO: 11, the HCDR2 includes an amino acid sequence as set forth in SEQ ID NO: 12, and the HCDR1 includes an amino acid sequence as set forth in SEQ ID NO: 13.

In some embodiments, the antibody or the antigen binding fragment thereof includes a LCDR3, a LCDR2, and a LCDR1, the LCDR3 includes an amino acid sequence as set forth in SEQ ID NO: 14, the LCDR2 includes an amino acid sequence as set forth in SEQ ID NO: 15, and the LCDR1 includes an amino acid sequence as set forth in SEQ ID NO: 16.

In some embodiments, the antibody or the antigen binding fragment thereof includes a HCDR3, a HCDR2, a HCDR1, a LCDR3, a LCDR2, and a LCDR1, the HCDR3 includes an amino acid sequence as set forth in SEQ ID NO: 11, the HCDR2 includes an amino acid sequence as set forth in SEQ ID NO: 12, the HCDR1 includes an amino acid sequence as set forth in SEQ ID NO: 13, the LCDR3 includes an amino acid sequence as set forth in SEQ ID NO: 14, the LCDR2 includes an amino acid sequence as set forth in SEQ ID NO: 15, and the LCDR1 includes an amino acid sequence as set forth in SEQ ID NO: 16.

In some embodiments, the antibody or the antigen binding fragment thereof includes a VH, and the VH may include an amino acid sequence as set forth in SEQ ID NO: 17. In some embodiments, the antibody or the antigen binding fragment thereof includes a VL, and the VL includes an amino acid sequence as set forth in SEQ ID NO: 18.

In some embodiments, the antibody or the antigen binding fragment thereof includes a VH and a VL, the VH includes an amino acid sequence as set forth in SEQ ID NO: 17 and the VL includes an amino acid sequence as set forth in SEQ ID NO: 18.

In some embodiments, the antibody or the antigen binding fragment thereof includes an amino acid sequence as set forth in SEQ ID NO: 19.

In some embodiments, the CAR includes an antigen binding domain, and the antigen binding domain includes an antibody targeting GPC-3 or an antigen binding fragment thereof.

In some embodiments, the antibody targeting GPC-3 or the antigen binding fragment thereof includes a HCDR3, and the HCDR3 includes an amino acid sequence as set forth in SEQ ID NO: 20; in some embodiments, the antibody or the antigen binding fragment thereof includes a HCDR2, and the HCDR2 includes an amino acid sequence as set forth in SEQ ID NO: 21; in some embodiments, the antibody or the antigen binding fragment thereof includes a HCDR1, and the HCDR1 includes an amino acid sequence as set forth in SEQ ID NO: 22. In some embodiments, the antibody or the antigen binding fragment thereof includes a LCDR3, and the LCDR3 includes an amino acid sequence as set forth in SEQ ID NO: 23; in some embodiments, the antibody or the antigen binding fragment thereof includes a LCDR2, and the LCDR2 includes an amino acid sequence as set forth in SEQ ID NO: 24; in some embodiments, the antibody or the antigen binding fragment thereof includes a LCDR1, and the LCDR1 includes an amino acid sequence as set forth in SEQ ID NO: 25.

In some embodiments, the antibody or the antigen binding fragment thereof includes a HCDR3, a HCDR2, and a HCDR1, the HCDR3 includes an amino acid sequence as set forth in SEQ ID NO: 20, the HCDR2 includes an amino acid sequence as set forth in SEQ ID NO: 21, and the HCDR1 includes an amino acid sequence as set forth in SEQ ID NO: 22.

In some embodiments, the antibody or the antigen binding fragment thereof includes a LCDR3, a LCDR2, and a LCDR1, the LCDR3 includes an amino acid sequence as set forth in SEQ ID NO: 23, the LCDR2 includes an amino acid sequence as set forth in SEQ ID NO: 24, and the LCDR1 includes an amino acid sequence as set forth in SEQ ID NO: 25.

In some embodiments, the antibody or the antigen binding fragment thereof includes a HCDR3, a HCDR2, a HCDR1, a LCDR3, a LCDR2, and a LCDR1, the HCDR3 includes an amino acid sequence as set forth in SEQ ID NO: 20, the HCDR2 includes an amino acid sequence as set forth in SEQ ID NO: 21, the HCDR1 includes an amino acid sequence as set forth in SEQ ID NO: 22, the LCDR3 includes an amino acid sequence as set forth in SEQ ID NO: 23, the LCDR2 includes an amino acid sequence as set forth in SEQ ID NO: 24, and the LCDR1 includes an amino acid sequence as set forth in SEQ ID NO: 25.

In some embodiments, the antibody or the antigen binding fragment thereof includes a VH, and the VH includes an amino acid sequence as set forth in SEQ ID NO: 26. In some embodiments, the antibody or the antigen binding fragment thereof includes a VL, and the VL includes an amino acid sequence as set forth in SEQ ID NO: 27.

In some embodiments, the antibody or the antigen binding fragment thereof includes a VH and a VL, the VH includes an amino acid sequence as set forth in SEQ ID NO: 26 and the VL includes an amino acid sequence as set forth in SEQ ID NO: 27.

In some embodiments, the antibody or the antigen binding fragment thereof includes an amino acid sequence as set forth in SEQ ID NO: 28.

In some embodiments, the antibody targeting GPC-3 or the antigen binding fragment thereof includes a HCDR3, and the HCDR3 includes an amino acid sequence as set forth in SEQ ID NO: 29; in some embodiments, the antibody or the antigen binding fragment thereof includes a HCDR2, and the HCDR2 includes an amino acid sequence as set forth in SEQ ID NO: 30; in some embodiments, the antibody or the antigen binding fragment thereof includes a HCDR1, and the HCDR1 includes an amino acid sequence as set forth in SEQ ID NO: 31. In some embodiments, the antibody or the antigen binding fragment thereof includes a LCDR3, and the LCDR3 includes an amino acid sequence as set forth in SEQ ID NO: 32; in some embodiments, the antibody or the antigen binding fragment thereof includes a LCDR2, and the LCDR2 includes an amino acid sequence as set forth in SEQ ID NO: 33; in some embodiments, the antibody or the antigen binding fragment thereof includes a LCDR1, and the LCDR1 includes an amino acid sequence as set forth in SEQ ID NO: 34.

In some embodiments, the antibody or the antigen binding fragment thereof includes a HCDR3, a HCDR2, and a HCDR1, the HCDR3 includes an amino acid sequence as set forth in SEQ ID NO: 29, the HCDR2 includes an amino acid sequence as set forth in SEQ ID NO: 30, and the HCDR1 includes an amino acid sequence as set forth in SEQ ID NO: 31.

In some embodiments, the antibody or the antigen binding fragment thereof includes a LCDR3, a LCDR2, and a LCDR1, the LCDR3 may include an amino acid sequence as set forth in SEQ ID NO: 32, the LCDR2 includes an amino acid sequence as set forth in SEQ ID NO: 33, and the LCDR1 includes an amino acid sequence as set forth in SEQ ID NO: 34.

In some embodiments, the antibody or the antigen binding fragment thereof includes a HCDR3, a HCDR2, a HCDR1, a LCDR3, a LCDR2, and a LCDR1, the HCDR3 includes an amino acid sequence as set forth in SEQ ID NO: 29, the HCDR2 includes an amino acid sequence as set forth in SEQ ID NO: 30, the HCDR1 includes an amino acid sequence as set forth in SEQ ID NO: 31, the LCDR3 includes an amino acid sequence as set forth in SEQ ID NO: 32, the LCDR2 includes an amino acid sequence as set forth in SEQ ID NO: 33, and the LCDR1 includes an amino acid sequence as set forth in SEQ ID NO: 34.

In some embodiments, the antibody or the antigen binding fragment thereof includes a VH, and the VH includes an amino acid sequence as set forth in SEQ ID NO: 35. In some embodiments, the antibody or the antigen binding fragment thereof includes a VL, and the VL includes an amino acid sequence as set forth in SEQ ID NO: 36.

In some embodiments, the antibody or the antigen binding fragment thereof includes a VH and a VL, the VH includes an amino acid sequence as set forth in SEQ ID NO: 35 and the VL includes an amino acid sequence as set forth in SEQ ID NO: 36.

In some embodiments, the antibody or the antigen binding fragment thereof includes an amino acid sequence as set forth in SEQ ID NO: 37.

In some embodiments, the antibody targeting GPC-3 or the antigen binding fragment thereof may include a HCDR3, and the HCDR3 includes an amino acid sequence as set forth in SEQ ID NO: 38; in some embodiments, the antibody or the antigen binding fragment thereof includes a HCDR2, and the HCDR2 includes an amino acid sequence as set forth in SEQ ID NO: 39; in some embodiments, the antibody or the antigen binding fragment thereof includes a HCDR1, and the HCDR1 includes an amino acid sequence as set forth in SEQ ID NO: 40. In some embodiments, the antibody or the antigen binding fragment thereof includes a LCDR3, and the LCDR3 includes an amino acid sequence as set forth in SEQ ID NO: 41; in some embodiments, the antibody or the antigen binding fragment thereof includes a LCDR2, and the LCDR2 includes an amino acid sequence as set forth in SEQ ID NO: 42; in some embodiments, the antibody or the antigen binding fragment thereof includes a LCDR1, and the LCDR1 includes an amino acid sequence as set forth in SEQ ID NO: 43.

In some embodiments, the antibody or the antigen binding fragment thereof includes a HCDR3, a HCDR2, and a HCDR1, the HCDR3 may include an amino acid sequence as set forth in SEQ ID NO: 38, the HCDR2 includes an amino acid sequence as set forth in SEQ ID NO: 39, and the HCDR1 includes an amino acid sequence as set forth in SEQ ID NO: 40.

In some embodiments, the antibody or the antigen binding fragment thereof includes a LCDR3, a LCDR2, and a LCDR1, the LCDR3 includes an amino acid sequence as set forth in SEQ ID NO: 41, the LCDR2 includes an amino acid sequence as set forth in SEQ ID NO: 42, and the LCDR1 includes an amino acid sequence as set forth in SEQ ID NO: 43.

In some embodiments, the antibody or the antigen binding fragment thereof includes a HCDR3, a HCDR2, a HCDR1, a LCDR3, a LCDR2, and a LCDR1, the HCDR3 includes an amino acid sequence as set forth in SEQ ID NO: 38, the HCDR2 includes an amino acid sequence as set forth in SEQ ID NO: 39, the HCDR1 includes an amino acid sequence as set forth in SEQ ID NO: 40, the LCDR3 includes an amino acid sequence as set forth in SEQ ID NO: 41, the LCDR2 includes an amino acid sequence as set forth in SEQ ID NO: 42, and the LCDR1 includes an amino acid sequence as set forth in SEQ ID NO: 43.

In some embodiments, the antibody or the antigen binding fragment thereof includes a VH, and the VH includes an amino acid sequence as set forth in SEQ ID NO: 44. In some embodiments, the antibody or the antigen binding fragment thereof includes a VL, and the VL includes an amino acid sequence as set forth in SEQ ID NO: 45.

In some embodiments, the antibody or the antigen binding fragment thereof includes a VH and a VL, the VH includes an amino acid sequence as set forth in SEQ ID NO: 44 and the VL includes an amino acid sequence as set forth in SEQ ID NO: 45.

In some embodiments, the antibody or the antigen binding fragment thereof includes an amino acid sequence as set forth in SEQ ID NO: 46.

In the present application, the antigen binding domain of the CAR may include an antibody specifically binding to CD20, CLDN18.2 or GPC-3 or an antigen binding fragment thereof. For example, the antibody of the present application or the antigen binding fragment thereof may include, but not limited to, a recombinant antibody, a monoclonal antibody, a human antibody, a humanized antibody, a chimeric antibody, a bispecific antibody, a single-chain antibody, a diabody, a triabody, a tetrabody, a Fv fragment, a scFv fragment, a Fab fragment, a Fab′fragment, a F(ab′)₂ fragment.

In the present application, the antigen binding fragment may include Fab, Fab′, F(ab′)₂, F (ab)₂, a Fv fragment, scFv, di-scFv, and/or dAb.

In the present application, the antigen binding domain may be a single-chain antibody. For example, the CD20 binding domain is scFv. The scFv may include a sequence as set forth in SEQ ID NO: 10. For example, the CD20 binding domain may include the VH, VL and linking peptide of the antibody. For example, the linking peptide includes an amino acid sequence as set forth in SEQ ID NO: 9.

For example, the CLDN18.2 binding domain is scFv. The scFv may include a sequence as set forth in SEQ ID NO: 19. For example, the CLDN18.2 binding domain may include the VH, VL and linking peptide of the antibody. For example, the linking peptide includes an amino acid sequence as set forth in SEQ ID NO: 9.

For example, the GPC-3 binding domain is scFv. The scFv may include a sequence as set forth in SEQ ID NO: 28. For example, the GPC-3 binding domain may include the VH, VL and linking peptide of the antibody. For example, the linking peptide includes an amino acid sequence as set forth in SEQ ID NO: 9.

For example, the GPC-3 binding domain is scFv. The scFv may include a sequence as set forth in SEQ ID NO: 37. For example, the GPC-3 binding domain may include the VH, VL and linking peptide of the antibody. For example, the linking peptide includes an amino acid sequence as set forth in SEQ ID NO: 9.

For example, the GPC-3 binding domain is scFv. The scFv may include a sequence as set forth in SEQ ID NO: 46. For example, the GPC-3 binding domain may include the VH, VL and linking peptide of the antibody. For example, the linking peptide includes an amino acid sequence as set forth in SEQ ID NO: 9.

In the present application, the CAR may include a transmembrane domain, and the transmembrane domain may include a transmembrane domain of a protein selected from the group consisting of: CD28, CD3e, CD45, CD5, CD8, CD9, CD16, CD22, CD33, CD37, CD64, CD80, CD86, CD134, CD137, and CD154. In some embodiments, the transmembrane domain includes an amino acid sequence as set forth in SEQ ID NO: 47.

In the present application, the CAR may include a co-stimulatory domain, and the co-stimulatory domain may include a co-stimulatory domain of a protein selected from the group consisting of: co-stimulatory signaling regions in CD28, 4-1BB, CD40L, TIM1, CD226, DR3, SLAM, ICOS, OX40, NKG2D, 2B4, CD244, FcϵRIγ, BTLA, CD27, CD30, GITR, HVEM, DAP10, CD2, NKG2C, LIGHT, and DAP12. In some embodiments, the co-stimulatory domain includes a co-stimulatory domain of 4-1BB. In some embodiments, the co-stimulatory domain includes an amino acid sequence as set forth in SEQ ID NO: 48.

In the present application, the CAR may include an intracellular signaling domain, and the intracellular signaling domain may include a signaling domain derived from CD3. For example, the intracellular signaling domain may include an amino acid sequence as set forth in SEQ ID NO: 49.

In the present application, the CAR may include, sequentially from N-terminus to C-terminus, an antigen binding domain, a transmembrane domain, a co-stimulatory domain, and an intracellular signaling domain. For example, the antigen binding domain may include an amino acid sequence as set forth in SEQ ID NO: 10, SEQ ID NO: 19, SEQ ID NO: 28, SEQ ID NO: 37, or SEQ ID NO: 46, the transmembrane domain may include an amino acid sequence as set forth in SEQ ID NO: 47, the co-stimulatory domain may include an amino acid sequence as set forth in SEQ ID NO: 48, and the intracellular signaling domain may include an amino acid sequence as set forth in SEQ ID NO: 49.

In the present application, the CAR may further include a hinge region, by which the antigen binding domain and the transmembrane domain are linked. For example, the hinge region is derived from IgG family. For example, the hinge region is derived from IgG1. For example, the hinge region is derived from IgG4. For example, the hinge region is derived from IgD. For example, the hinge region is derived from CD8. For example, the hinge region may include an amino acid sequence as set forth in SEQ ID NO: 50.

In the present application, the CAR may be further linked to a signal peptide. For example, the signal peptide may be derived from CD8. For example, the signal peptide may be CD8a. The signal peptide is linked to the N-terminus of the antigen binding domain.

In the present application, the immune effector cell may further include and/or express a Bcl-2 protein or a functionally active fragment thereof. In some embodiments, the Bcl-2 protein or the functionally active fragment thereof includes an amino acid sequence as set forth in SEQ ID NO: 52.

In some embodiments, the Bcl-2 protein is introduced exogenously, for example, by adding a cleavage peptide and a sequence encoding the Bcl-2 protein or a functionally active fragment thereof to the nucleotide sequence encoding the chimeric antigen receptor, so that the Bcl-2 protein or the functionally active fragment thereof is expressed in the immune effector cell.

In some embodiments, for the modified immune effector cell, the modification of the immune effector cell includes making the immune effector cell include and/or express a chimeric antigen receptor and introducing an exogenous Bcl-2 protein or a functionally active fragment thereof into the immune effector cell.

In the present application, the functionally active fragment of Bcl-2 includes a fragment enabling the Bcl-2 protein to exert antiapoptotic function. For example, the functionally active fragment of Bcl-2 protein includes the BH4 domain thereof.

In some embodiments, the BBZ includes a hinge region, a transmembrane domain, a co-stimulatory domain, and an intracellular signaling domain.

In some embodiments, the 20BBZ includes a sequence of CAR targeting CD20. The 20BBZ includes an amino acid sequence as set forth in SEQ ID NO: 54. The 20BBZ-Bcl-2 includes sequences capable of expressing the CAR targeting CD20 and Bcl-2 protein or functionally active fragments thereof. The 20BBZ-Bcl-2 includes an amino acid sequence as set forth in SEQ ID NO: 53.

In some embodiments, the Ab10BBZ includes a sequence of CAR targeting CLDN18.2. The Ab10BBZ includes an amino acid sequence as set forth in SEQ ID NO: 56. The Ab10BBZ-Bcl-2 includes sequences capable of expressing the CAR targeting CLDN18.2 and Bcl-2 protein or functionally active fragments thereof. The Ab10BBZ-Bcl-2 includes an amino acid sequence as set forth in SEQ ID NO: 55.

In some embodiments, the GC33BBZ includes a sequence of CAR targeting GPC-3. The GC33BBZ includes an amino acid sequence as set forth in SEQ ID NO: 58. The GC33BBZ-Bcl-2 includes sequences capable of expressing the CAR targeting GPC-3 and Bcl-2 protein or functionally active fragments thereof. The GC33BBZ-Bcl-2 includes an amino acid sequence as set forth in SEQ ID NO: 57.

In some embodiments, the GC179BBZ includes a sequence of CAR targeting GPC-3. The GC179BBZ includes an amino acid sequence as set forth in SEQ ID NO: 60. The GC179BBZ-Bcl-2 includes sequences capable of expressing the CAR targeting GPC-3 and Bcl-2 protein or functionally active fragments thereof. The GC179BBZ-Bcl-2 includes an amino acid sequence as set forth in SEQ ID NO: 59.

In some embodiments, the M3C11BBZ includes a sequence of CAR targeting GPC-3. The M3C11BBZ includes an amino acid sequence as set forth in SEQ ID NO: 62. The M3C11BBZ-Bcl-2 includes sequences capable of expressing the CAR targeting GPC-3 and Bcl-2 protein or functionally active fragments thereof. The M3C11BBZ-Bcl-2 includes an amino acid sequence as set forth in SEQ ID NO: 61.

In the present application, the CAR includes an amino acid sequence as set forth in any one of SEQ ID NO: 53, SEQ ID NO: 55, SEQ ID NO: 57, SEQ ID NO: 59, and SEQ ID NO: 61.

Nucleic Acid Molecule, Vector, Cell, Preparation Method, Composition

In another aspect, the present application further provides one or more isolated nucleic acid molecules, which may encode the CAR of the present application. The one or more isolated nucleic acid molecules of the present application may be isolated nucleotides, deoxyribonucleotides or ribonucleotides of any length, or analogues thereof isolated from their natural environment or synthesized artificially, but they can encode the CAR of the present application.

In the present application, the nucleic acid molecules encode the chimeric antigen receptor and the Bcl-2 protein or the functionally active fragment thereof.

In the present application, the nucleic acid molecules also include a nucleotide sequence encoding the cleavage peptide. For example, the cleavage peptide may be 2A peptide. For example, the cleavage peptide may be selected from one or more of P2A, T2A, E2A, and F2A. For example, the cleavage peptide may include an amino acid sequence as set forth in SEQ ID NO: 51.

In some embodiments, the nucleic acid molecules include, sequentially from 5′ end to 3′ end, a sequence encoding the antigen binding domain, a sequence encoding the transmembrane domain, a sequence encoding the co-stimulatory domain, a sequence encoding the intracellular signaling domain, a sequence encoding the 2A peptide, and a sequence encoding the Bcl-2 protein or the functionally active fragment thereof.

For example, the nucleic acid molecules include, sequentially from 5′ end to 3′ end, nucleotide sequences encoding scFv targeting CD20, 4-1BB, CD3ζ, 2A, and Bcl-2. The nucleic acid sequences of the various moieties may be linked directly or indirectly. The indirect linkage may be through linkers.

For example, the nucleic acid molecules include, sequentially from 5′ end to 3′ end, nucleotide sequences encoding scFv targeting CLDN18.2, 4-1BB, CD3ζ, 2A, and Bcl-2. The nucleic acid sequences of the various moieties may be linked directly or indirectly. The indirect linkage may be through linkers.

For example, the nucleic acid molecules include, sequentially from 5′ end to 3′ end, nucleotide sequences encoding scFv targeting GPC-3, 4-1BB, CD3ζ, 2A, and Bcl-2. The nucleotide sequences of the various moieties may be linked directly or indirectly. The indirect linkage may be through linkers. For example, the nucleotide sequence encoding the scFv targeting GPC-3 may be the nucleotide sequence encoding GC33 scFv, GC179 scFv or M3C11scFv as provided in the embodiments.

In another aspect, the present application further provides a vector which may include the nucleic acid molecule of the present application. The vector can make the genetic elements it carries be expressed in a host cell by transforming, transducing or transfecting the host cell. For example, the vector may include plasmids; phagemids; Cosmids; artificial chromosomes, such as yeast artificial chromosomes (YACs), bacterial artificial chromosomes (BACs) or P1-derived artificial chromosomes (PACs); phages, such as lambda phages or M13 phages and animal viruses, and the like. The species of animal viruses used as the vector are retrovirus (including lentivirus), adenovirus, adeno-associated virus, herpes virus (e.g., herpes simplex virus), poxvirus, baculovirus, papilloma virus, papovavirus (e.g., SV40). Further for example, the vector may contain various elements for controlling the expression, including promoter sequences, transcription initiation sequences, enhancer sequences, selective elements, and reporter genes. In addition, the vector may also contain replication initiation sites. Moreover, the vector may also include ingredients that help its entry into cells, such as virion, lipidosome, or protein coat, but not only these substances. For example, the vector of the present application may be selected from one or more of plasmids, retroviral vectors, and lentiviral vectors. For example, the vector of the present application may be lentiviral vectors.

In another aspect, the present application further provides an immune effector cell, which may include the nucleic acid molecule of the present application or the vector of the present application. The cells may include the progeny of a single cell. Due to natural, accidental, or intentional mutations, the progeny may not necessarily be exactly the same as the original parent cells (in the form of the total DNA complement or in the genome). For example, the cells may also be mammal cells. In some embodiments, the immune effector cells also include T lymphocytes, such as α/β T lymphocytes and γ/δ T lymphocytes. For example, the immune effector cells may be derived from human PBMCs. For example, the T lymphocytes may be CD4+T cells or CD8+T cells. In some embodiments, the immune effector cells include natural killer cells, natural killer T cells, mastocytes, and bone marrow-derived phagocytes.

In another aspect, the present application further provides a method for preparing the immune effector cell of the present application, which may include introducing the isolated nucleic acid molecule of the present application or the vector of the present application into the immune effector cell.

In another aspect, the present application further provides a composition, which may include the immune effector cell of the present application. In some embodiments, the composition also includes a optionally pharmaceutically acceptable carrier.

In some embodiments, the acceptable ingredients of the composition may be non-toxic to the recipient at the dosage and concentration used. The pharmaceutical composition of the present application includes, but not limited to, liquid, frozen and freeze-dried compositions.

In some embodiments, the pharmaceutically acceptable adjuvant includes any and all solvents, dispersion media, isotonic agents, and absorption delaying agents that are compatible with the immune effector cell, which are generally safe, non-toxic and neither biologically nor otherwise undesirable.

In some embodiments, the composition is administered parenterally, transdermally, intraperitoneally, intra-arterially, intrathecally and/or intranasally or directly injected into tissues. For example, the composition may be administered to patients or subjects by means of infusion or injection. In some embodiments, the administration of the pharmaceutical composition can be carried out in different ways, such as intravenous, intraperitoneal, subcutaneous, intramuscular, topical, or intradermal administration. In some embodiments, the composition is administered incessantly. The incessant (or continuous) administration can be achieved by a small pump system worn by the patient to measure the therapeutic agent flowing into the patient, as described in WO2015/036583.

Use and Application

In another aspect, the present application further provides use of the immune effector cell, the nucleic acid molecule, the vector, the composition in the preparation of drugs for preventing and/or treating diseases and/or disorders.

In another aspect, the present application further provides a method for preventing and/or treating diseases and/or disorders, the method may include administering to a subject in need the immune effector cell or composition of the present application.

In the present application, the subject may include human and non-human animals. For example, the subject may include, but not limited to, cat, dog, horse, pig, cow, sheep, rabbit, mouse, rat, or monkey.

In another aspect, the immune effector cell of the present application, the nucleic acid molecule of the present application, the vector of the present application and/or the composition of the present application, they may be used for preventing, relieving or treating tumors.

In some embodiments, the diseases and/or disorders are associated with the expression of CD20.

In some embodiments, the diseases and/or disorders are associated with the expression of CLDN18.2.

In some embodiments, the diseases and/or disorders are associated with the expression of GPC-3.

In some embodiments, the diseases and/or disorders include tumors.

In some embodiments, the tumors include solid tumors and/or blood tumors.

In some embodiments, the tumors include CD20 positive tumors.

In some embodiments, the tumors include CLDN18.2 positive tumors.

In some embodiments, the tumors include GPC-3 positive tumors.

In some embodiments, the tumors include lymphoma.

In some embodiments, the tumors include pancreatic cancer.

Without intending to be limited by any theory, the embodiments below are intended only to illustrate the immune effector cell, the preparation method and the use of the present application and are not intended to limit the inventive scope of the present application.

### EMBODIMENTS

### Embodiment 1. Preparation of Anti-CD20 CAR-T Cells, Anti-CLDN18.2 CAR-T Cells, and Anti-GPC-3 CAR-T Cells

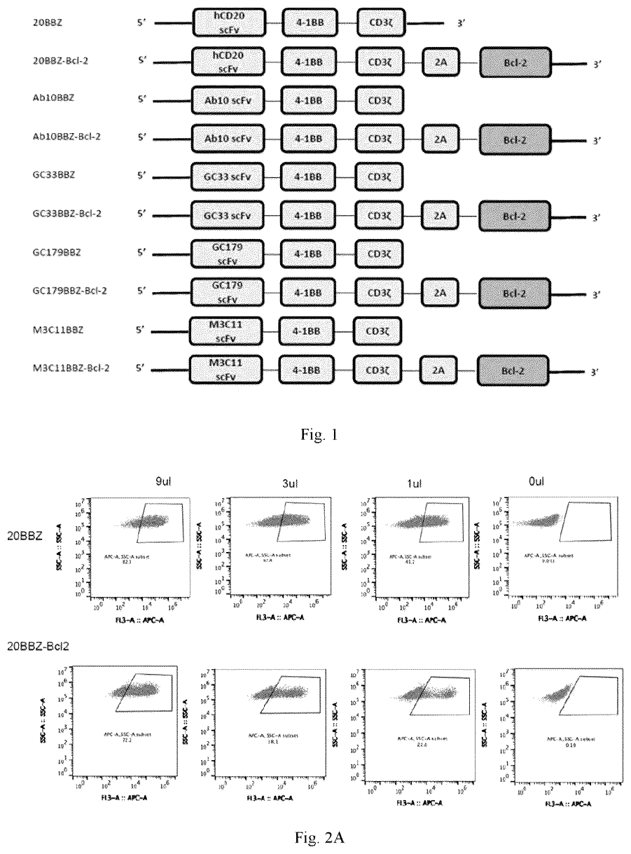

CAR targeting CD20 (20BBZ), CAR targeting CLDN18.2 (Ab10BBZ) and CARs targeting GPC-3 (GC33BBZ, GC179BBZ and M3C11BBZ) were prepared, wherein the structures of the CARs were as shown in FIG. 1. The following sequences were artificially synthesized: scFv 20, scFv Abl 0, scFv GC33, scFv GC179, scFv M3C11, a hinge region, a transmembrane domain, a 4-1BB costimulatory domain, a CD3 intracellular signaling domain. Of those, BBZ can be obtained by connecting the hinge region, the transmembrane domain, the 4-1BB costimulatory domain and the CD3 intracellular signaling domain end to end. To both ends of scFv 20 which specifically binds to CD20, scFv Abl 0 which specifically binds to CLDN18.2, scFv GC33, scFv GC179 and scFv M3C11 which specifically bind to GPC-3, and BBZ was added XbaI and BamHI restriction sites to clone pCDH-MSCVEF vectors by overlap PCR. PCR amplification was performed, and to the 5′ end was added XbaI restriction sites (containing protected bases), scFv 20/scFv Ab10/scFv GC33/scFv scFv GC179/scFv M3C11, a hinge region, a transmembrane domain, a 4-1BB costimulatory domain, a CD3 intracellular signaling domain, and a BamHI restriction site by extension PCR so that the PCR amplification resulted in the CARs: 20BBZ, Ab10BBZ, GC33BBZ, GC179BBZ and M3C11BBZ.

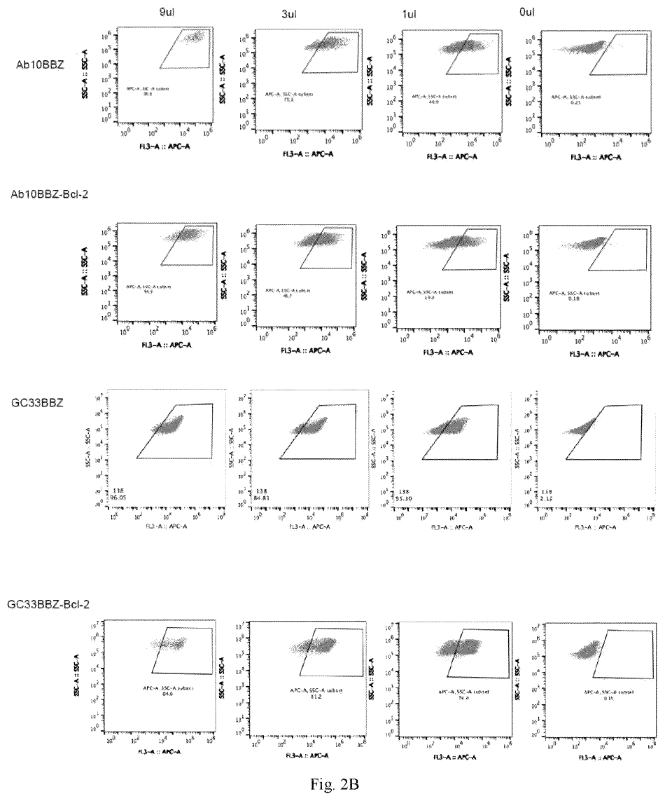

Viruses containing anti-CD20 CAR (20BBZ viruses), viruses containing anti-CLDN18.2 CAR (Ab10BBZ viruses) and viruses containing anti-GPC-3 CAR (GC33BBZ viruses, GC179BBZ viruses and M3C11BBZ viruses) were prepared. The correctly sequenced clones were subjected to non-endotoxin extraction with NucleoBond Xtra Midi Plus EF Kit and used to co-transfect 293 cells along with lentivirus packaging plasmids (VSV-g, pMD Gag/Pol or RSV-REV). After culture at 37° C., 5% CO₂ for 48 hrs., the supernatant was collected, filtered at 0.45 μM, and centrifuged at 25,000 RPM using Beckman Ultra-Centrifuge and SW28 Rotor for 2 hrs. to concentrate the viruses, that are viruses containing pCDH-MSCVEF-20BBZ, viruses containing pCDH-MSCVEF-Ab10BBZ, viruses containing pCDH-MSCVEF-GC33BBZ, viruses containing pCDH-MSCVEF-GC179BBZ and viruses containing pCDH-MSCVEF-M3C11BBZ (abbreviated as 20BBZ viruses, Ab10BBZ viruses, GC33BBZ viruses, GC179BBZ viruses, and M3C11BBZ viruses) for use in subsequent production of CAR-T cells. 293 cells were infected by the resultant viruses and detected by flow cytometry using anti-mouse Fab antibodies (Jackson ImmunoResearch #115-605-006) for virus titer. FIG. 2 shows the results of flow cytometry by adding 1 μL, 3 μL or 9 μL of the viruses, with no virus added as blank control. The results show that the CAR expression levels of CARs: 20BBZ, Ab10BBZ, GC33BBZ, GC179BBZ and M3C11BBZ increase with the increase of added virus doses.

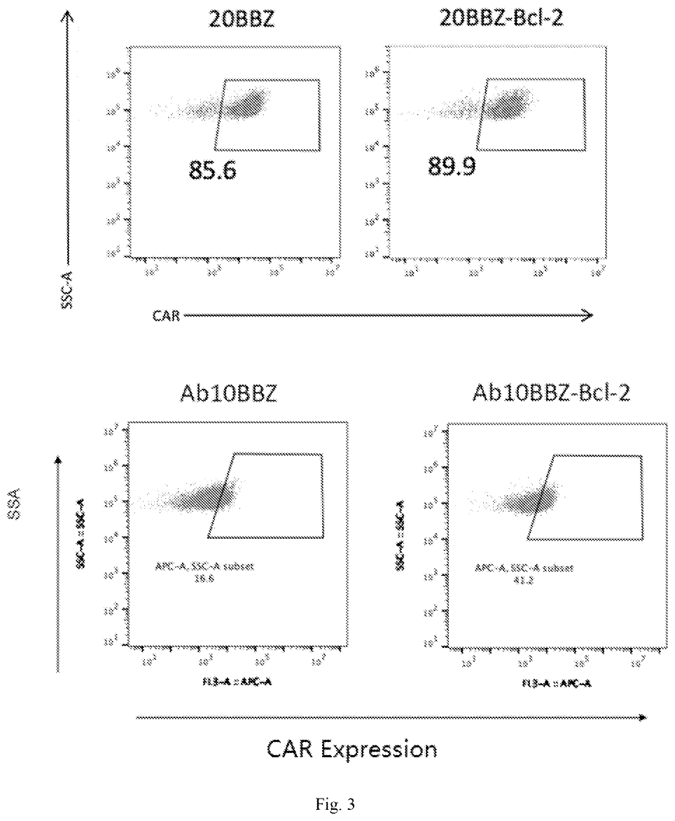

Anti-CD20 CAR-T cells (20BBZ CAR-T cells), anti-CLDIN18.2CAR-T cells (Ab10BBZ CAR-T cells) and anti-GPC-3CAR-T cells (GC33BBZ CAR-T cells, GC179BBZ CAR-T cells and M3C11BBZ CAR-T cells) were prepared. Human PBMCs were purified by Stemcell T cell isolation kit (purchased from stem cell Catlog #19671), and then inoculated into 96-well plates coated with anti-hCD3 (purchased from Bioxcell #BE0001-2) and anti-hCD28 (purchased from Bioxcell #BE0248). After 2 days, the cells were infected with the 20BBZ viruses, Ab10BBZ viruses, GC33BBZ viruses, GC179BBZ viruses and M3C11BBZ viruses prepared in this Embodiment at MOI (multiplicity of infection, i.e., a ratio of virus amount to cell number)=10-20. After 1 day, the cell culture was continued by replacing the medium, and the medium was 10% FBS-containing RPMI complete medium, IL2 (50 IU/ml), and IL21 (4 ng/ml). The culture was stimulated by artificial antigen-presenting cells (Raji-CLDN18.2 cells irradiated by X-ray at 100 Gray) or anti-hCD3 (0.1 μg/ml) or anti-hCD28 (0.25 μg/ml) every 6 days. After 2 rounds of stimulation, the resultant cells were 20BBZ CAR-T cells, Ab10BBZ CAR-T cells, GC33BBZ CAR-T cells, GC179BBZ CAR-T cells and M3C11BBZ CAR-T cells. By using Alexa Fluor® 647 AffiniPure F(ab′)2 Fragment Goat Anti-Mouse IgG, Fab fragment specific antibody staining and flow cytometry, the results are shown in FIG. 3. The results show that the resultant cells are all CAR-positive.

### Embodiment 2. Preparation of Anti-CD20 Bcl-2 CAR-T Cells, Anti-CLDN18.2 Bcl-2 CAR-T Cells and Anti-GPC-3 Bcl-2 CAR-T Cells

Bcl-2-expressing CAR targeting CD20 (20BBZ-Bcl-2, with structure shown in FIG. 1), CAR targeting CLDN18.2 (Ab10BBZ-Bcl-2, with structure shown in FIG. 1), CARs targeting GPC-3 (GC33BBZ-Bcl-2, GC179BBZ-Bcl-2 and M3C11BBZ-Bcl-2, with structures shown in FIG. 1), and Bcl-2-expressing anti-CD20 CAR-T viruses (20BBZ-Bcl-2 viruses), anti-CLDN18.2 CAR-T viruses (Ab10BBZ-Bcl-2 viruses), and anti-GPC-3 CAR-T viruses (GC33BBZ-Bcl-2 viruses, GC179BBZ-Bcl-2 viruses and M3C11BBZ-Bcl-2 viruses) were prepared in accordance with the method of Embodiment 1.

The 20BBZ, Ab10BBZ, GC33BBZ, GC179BBZ and M3C11BBZ were subjected to stop codon removal, and ligated to fragments of 2A, Bcl-2, and then subjected to overlap PCR, molecular cloning, and virus production to give pCDH-MSCVEF-20BBZ-Bcl-2 viruses, pCDH-MSCVEF-Ab10BBZ-Bcl-2 viruses, pCDH-MSCVEF-GC33BBZ-Bcl-2 viruses, pCDH-MSCVEF-GC179BBZ-Bcl-2 viruses, and pCDH-MSCVEF-M3C11BBZ-Bcl-2 viruses (abbreviated as 20BBZ-Bcl-2 viruses, Ab10BBZ-Bcl-2 viruses, GC33BBZ-Bcl-2 viruses, GC179BBZ-Bcl-2 viruses and M3C11BBZ-Bcl-2 viruses). The virus titer was detected by flow cytometry as in Embodiment 1. FIG. 2 shows the results of flow cytometry by adding 1 μL, 3 μL or 9 μL of the viruses, with no virus added as blank control. The results show that the CAR expression levels of the CARs: 20BBZ-Bcl-2, Ab10BBZ-Bcl-2, GC33BBZ-Bcl-2, GC179BBZ-Bcl-2, and M3C11BBZ-Bcl-2 increase with the increase of added virus doses.

Bcl-2-expressing anti-CD20 CAR-T cells (20BBZ-Bcl-2 CAR-T cells), anti-CLDIN18.2 CAR-T cells (Ab10BBZ-Bcl-2 CAR-T cells) and anti-GPC-3CAR-T cells (GC33BBZ-Bcl-2 CAR-T cells, GC179BBZ-Bcl-2 CAR-T cells and M3C11BBZ-Bcl-2 CAR-T cells) were prepared. Human PBMC-derived T cells were purified, activated, infected with 20BBZ-Bcl-2 viruses, Ab10BBZ-Bcl-2 viruses, GC33BBZ-Bcl-2 viruses, GC179BBZ-Bcl-2 viruses, M3C11BBZ-Bcl-2 viruses, and amplified to give 20BBZ-Bcl-2 CAR-T cells, Ab10BBZ-Bcl-2 CAR-T cells, GC33BBZ-Bcl-2 CAR-T cells, GC179BBZ-Bcl-2 CAR-T cells and M3C11BBZ-Bcl-2 CAR-T cells, respectively, which were stained with Alexa Fluor® 647 AffiniPure F(ab)₂ Fragment Goat Anti-Mouse IgG, Fab fragment specific antibodies by flow cytometry, with the results shown in FIG. 3. The results show that the resultant cells are all CAR positive.

### Embodiment 3. Expansion Ability of Anti-CD20 Bcl-2 CAR-T Cells

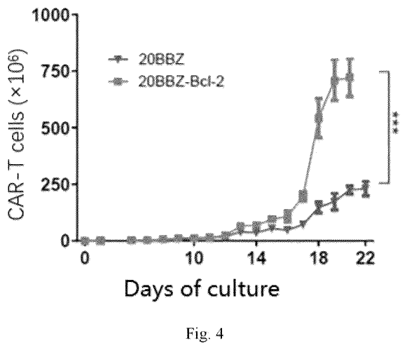

The CD20BBZ CAR-T cells prepared in Embodiment 1 and the CD20BBZ-Bcl-2 CAR-T cells prepared in Embodiment 2 were continuously cultured and stimulated with artificial antigen-presenting cells every 6 days. The cells were counted, with the results shown in FIG. 4. It can be seen from FIG. 4 that the CD20BBZ-Bcl-2 CAR-T cells have better expansion ability as compared with the CD20BBZ CAR-T cells.

### Embodiment 4. Expansion Ability of Anti-CLDN18.2 Bcl-2 CAR-T Cells

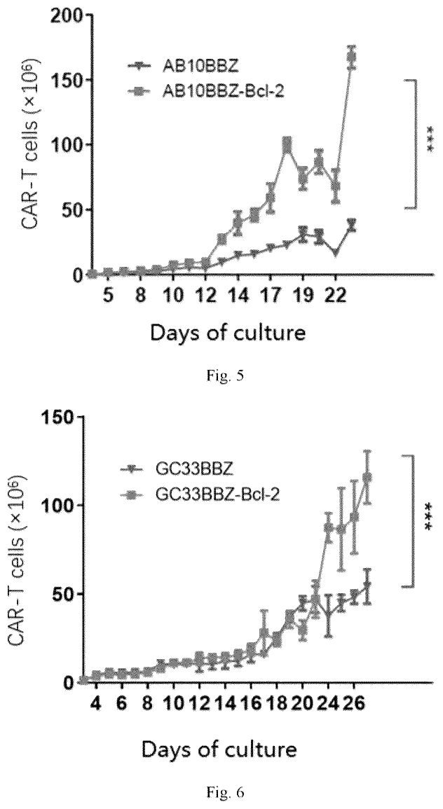

The Ab10BBZ CAR-T cells prepared in Embodiment 1 and the Ab10BBZ-Bcl-2 CAR-T cells prepared in Embodiment 2 were continuously cultured and stimulated with artificial antigen-presenting cells every 6 days. The cells were counted, with the results shown in FIG. 5. It can be seen from FIG. 5 that the Ab10BBBZ-Bcl-2 CAR-T cells have better in vitro expansion ability as compared with the Ab10BBZ CAR-T cells.

### Embodiment 5. Expansion Ability of Anti-GPC-3 Bcl-2 CAR-T Cells

The GC33BBZ CAR-T cells prepared in Embodiment 1 and the GC33BBZ-Bcl-2 CAR-T cells prepared in Embodiment 2 were continuously cultured and stimulated with artificial antigen-presenting cells every 6 days. The cells were counted, with the results shown in FIG. 6. It can be seen from FIG. 6 that the GC33BBZ-Bcl-2 CAR-T cells have better in vitro expansion ability as compared with the GC33BBZ CAR-T cells.

### Embodiment 6. In Vitro Tumor Killing Ability of Anti-CD20 Bcl-2 CAR-T Cells

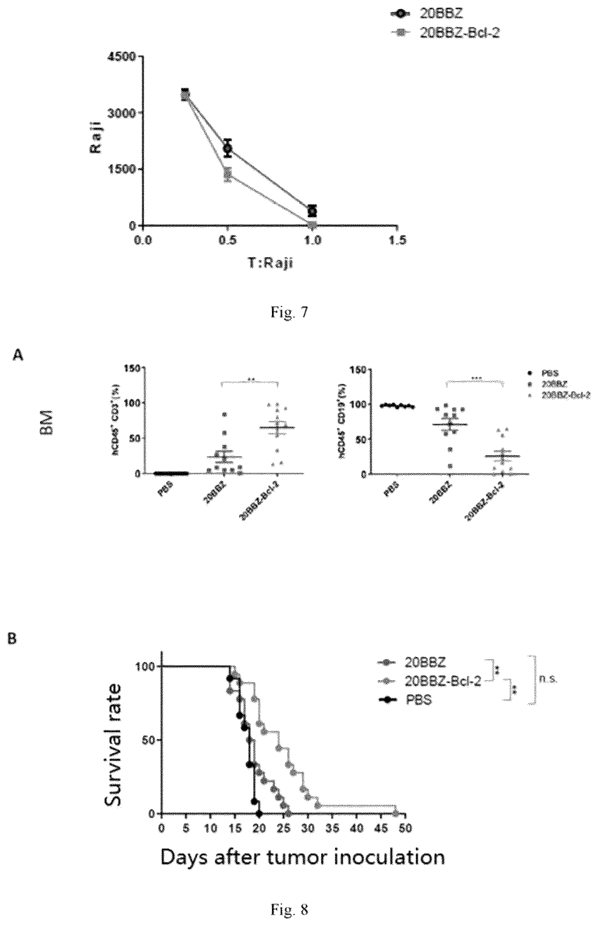

The 20BBZ CAR-T cells prepared in Embodiment 1 and the 20BBZ-Bcl-2 CAR-T cells prepared in Embodiment 2 were inoculated into 96-well plates, and CD20-positive tumor cells (Raji) were added at a CAR-T: tumor cell ratio of 1:1, 0.5:1, and 0.25:1. After 24 hrs., the survival of Raji was detected by flow cytometer. As show in the detection of in vitro tumor killing effect in FIG. 7, the 20BBZ-Bcl-2 CAR-T cells have a similar in vitro tumor killing ability as the 20BBZ CAR-T cells.

### Embodiment 7. In Vivo Tumor Killing Ability of Anti-CD20 Bcl-2 CAR-T Cells

3×10⁶ Raji cells were inoculated into B-NDG mice by intravenous injection. After 6 days, the mice were administered with 10⁷ CD20BBZ CAR-T cells or CD20BBZ-Bcl-2 CAR-T cells for treatment, and PBS was given as blank control. The ratio of T cells to Raji cells in the bone marrow of mice was measured, with the results shown in FIG. 8A, respectively. In the bone marrow, the ratio of CD20BBZ-Bcl-2 CAR-T cells to CD20BBZ CAR-T cells was significantly statistically different (P<0.01). As compared with the control group of CD20BBZ CAR-T cell treatment, the CD20BBZ-Bcl-2 CAR-T cell treatment significantly reduced the proportion of Raji cells in the bone marrow, which was significantly statistically different (P<0.001).

3×10⁶ Raji cells were inoculated into B-NDG mice by intravenous injection. After 6 days, the mice were administered with 10⁷ CD20BBZ CAR-T cells or CD20BBZ-Bcl-2 CAR-T cells for treatment, and PBS was given as blank control. The survival curve of mice was plotted, with the results shown in FIG. 8B. As compared with the control group of CD20BBZ CAR-T cell treatment, the mice treated with CD20BBZ-Bcl-2 CAR-T cells had a longer survival time of up to 48 days, demonstrating that the CD20BBZ-Bcl-2 CAR-T cells have significant anti-tumor ability in vitro.

### Embodiment 8. In Vivo Tumor Killing Ability of Anti-CLDN18.2 Bcl-2 CAR-T Cells

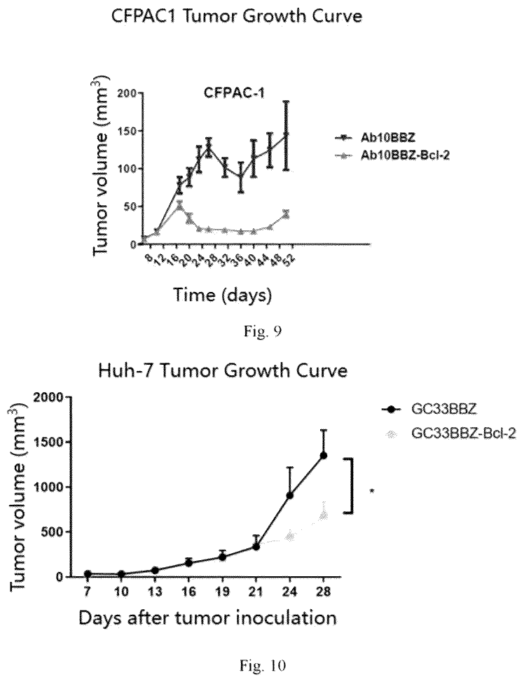

3×10⁶ CFPAC-1 tumor cells were subcutaneously inoculated into B-NDG mice. After 6 days, the mice were administered with 10⁷ Ab10BBZ CAR-T cells or Ab10BBZ-Bcl-2 CAR-T cells for treatment. The mice were measured for tumor load, with the results shown in FIG. 9. As compared with Ab10BBZ CAR-T cells, the Ab10BBZ-Bcl-2 CAR-T cells can control the tumor load better. The results show that the tumor of mice treated with Ab10BBZ-Bcl-2 CAR-T cells began to shrink on Day 16, while the control tumor treated with Ab10BBZ CAR-T cells kept growing rapidly, demonstrating that the Ab10BBZ-Bcl-2 CAR-T cells had obvious anti-tumor ability in vivo.

### Embodiment 9. In Vivo Tumor Killing Ability of Anti-GPC-3 Bcl-2 CAR-T Cells

3×10⁶ Huh-7 tumor cells were subcutaneously inoculated into B-NDG mice. After 7 days, the mice were administered with 10⁷ GC33BBZ CAR-T cells or GC33BBZ-Bcl-2 CAR-T cells for treatment. The mice were measured for tumor load, with the results shown in FIG. 10. As compared with GC33BBZ CAR-T cells, the GC33BBZ-Bcl-2 CAR-T cells can control the tumor load better and inhibit the tumor growth, demonstrating that the GC33BBZ-Bcl-2 CAR-T cells had obvious anti-tumor ability in vivo.

1. An immune effector cell, comprising and/or expressing a chimeric antigen receptor (CAR), and a Bcl-2 protein or a functionally active fragment thereof.

2. The immune effector cell according to claim 1, wherein the CAR comprises an antigen binding domain, and the antigen binding domain comprises an antibody specifically binding to CD20 or an antigen binding fragment thereof.

3. The immune effector cell according to claim 2, wherein the antibody or the antigen binding fragment thereof comprises a HCDR1, HCDR2, HCDR3, LCDR1, LCDR2, and LCDR3,
wherein the HCDR1 comprises an amino acid sequence as set forth in SEQ ID NO: 3,
wherein the HCDR2 comprises an amino acid sequence as set forth in SEQ ID NO: 2,
wherein the HCDR3 comprises an amino acid sequence as set forth in SEQ ID NO: 1,
wherein the LCDR1 comprises an amino acid sequence as set forth in SEQ ID NO: 6,
wherein the LCDR2 comprises an amino acid sequence as set forth in SEQ ID NO: 5, and
wherein the LCDR3 comprises an amino acid sequence as set forth in SEQ ID NO: 4.

4-8. (canceled)

9. The immune effector cell according to claim 2, wherein the antibody or the antigen binding fragment thereof comprises a VH and a VL,
wherein the VH comprises an amino acid sequence as set forth in SEQ ID NO: 7, and
wherein the VL comprises an amino acid sequence as set forth in SEQ ID NO: 8.

10-11. (canceled)

12. The immune effector cell according to claim 1, wherein the CAR comprises an antigen binding domain, and the antigen binding domain comprises an antibody specifically binding to CLDN18.2 or an antigen binding fragment thereof.

13. The immune effector cell according to claim 12, wherein the antibody or the antigen binding fragment thereof comprises a HCDR1, HCDR2, HCDR3, LCDR1, LCDR2, and LCDR3,
wherein the HCDR1 comprises an amino acid sequence as set forth in SEQ ID NO: 13,
wherein the HCDR2 comprises an amino acid sequence as set forth in SEQ ID NO: 12,
wherein the HCDR3 comprises an amino acid sequence as set forth in SEQ ID NO: 11,
wherein the LCDR1 comprises an amino acid sequence as set forth in SEQ ID NO: 16,
wherein the LCDR2 comprises an amino acid sequence as set forth in SEQ ID NO: 15, and
wherein the LCDR3 comprises an amino acid sequence as set forth in SEQ ID NO: 14.

14-18. (canceled)

19. The immune effector cell according to claim 12, wherein the antibody or the antigen binding fragment thereof comprises a VH and a VL,
wherein the VH comprises an amino acid sequence as set forth in SEQ ID NO: 17, and
wherein the VL comprises an amino acid sequence as set forth in SEQ ID NO: 18.

20-21. (canceled)

22. The immune effector cell according to claim 1, wherein the CAR comprises an antigen binding domain, and the antigen binding domain comprises an antibody or an antigen binding fragment thereof specifically binding to GPC-3.

23. The immune effector cell according to claim 22, wherein the antibody or the antigen binding fragment thereof comprises a HCDR1, HCDR2, HCDR3, LCDR1, LCDR2, and LCDR3,
wherein the HCDR1 comprises an amino acid sequence as set forth in SEQ ID NO: 22, SEQ ID NO: 31, and SEQ ID NO: 40,
wherein the HCDR2 comprises an amino acid sequence as set forth in SEQ ID NO: 21, SEQ ID NO: 30, and SEQ ID NO: 39,
wherein the HCDR3 comprises an amino acid sequence as set forth in SEQ ID NO: 20 SEQ ID NO: 29, and SEQ ID NO: 38,
wherein the LCDR1 comprises an amino acid sequence as set forth in SEQ ID NO: 25, SEQ ID NO: 34, and SEQ ID NO:43,
wherein the LCDR2 comprises an amino acid sequence as set forth in SEQ ID NO: 24, SEQ ID NO: 33, and SEQ ID NO: 42, and
wherein the LCDR3 comprises an amino acid sequence as set forth in SEQ ID NO: 23, SEQ ID NO: 32 and SEQ ID NO: 41.

24-28. (canceled)

29. The immune effector cell according to claim 22, wherein the antibody or the antigen binding fragment thereof comprises a VH and a VL,
wherein the VH comprises an amino acid sequence as set forth in any one of SEQ ID NO: 26, SEQ ID NO: 35, and SEQ ID NO: 44, and
wherein the VL comprises an amino acid sequence as set forth in any one of SEQ ID NO: 27, SEQ ID NO: 36, and SEQ ID NO: 45.

30-31. (canceled)

32. The immune effector cell according to claim 2, wherein the antibody comprises a single-chain antibody.

33. The immune effector cell according to claim 1, wherein the CAR comprises a transmembrane domain, and the transmembrane domain comprises a transmembrane domain derived from a protein selected from the group consisting of: CD28, CD3e, CD45, CD5, CD8, CD9, CD16, CD22, CD33, CD37, CD64, CD80, CD86, CD134, CD137, and CD154.

34. (canceled)

35. The immune effector cell according to claim 1, wherein the CAR comprises a co-stimulatory domain, and the co-stimulatory domain comprises one or more co-stimulatory domains of a protein selected from the group consisting of: co-stimulatory signaling regions in CD28, 4-1BB, CD40L, TIM1, CD226, DR3, SLAM, ICOS, OX40, NKG2D, 2B4, CD244, FcϵRIγ, BTLA, CD27, CD30, GITR, HVEM, DAP10, CD2, NKG2C, LIGHT, and DAP12.

36. (canceled)

37. The immune effector cell according to claim 1, wherein the CAR comprises an intracellular signaling domain, and the intracellular signaling domain comprises an intracellular signaling domain derived from CD3ζ.

38. (canceled)

39. The immune effector cell according to claim 1, wherein the CAR comprises a hinge region, and the hinge region is located between the antigen binding domain and the transmembrane domain.

40-41. (canceled)

42. The immune effector cell according to claim 1, wherein the CAR comprises an amino acid sequence as set forth in any one of SEQ ID NO: 53, SEQ ID NO: 55, SEQ ID NO: 57, SEQ ID NO: 59, and SEQ ID NO: 61.

43. The immune effector cell according to claim 1, wherein the Bcl-2 protein or the functionally active fragment thereof is an exogenous Bcl-2 protein or a functionally active fragment thereof.

44. The immune effector cell according to claim 1, wherein the immune effector cell comprises T cells.

45. The immune effector cell according to claim 1, wherein the Bcl-2 protein or the functionally active fragment thereof comprises an amino acid sequence as set forth in SEQ ID NO: 52.

46. A nucleic acid molecule encoding the CAR and the Bcl-2 protein or the functionally active fragment thereof of claim 1.

47. The nucleic acid molecule according to claim 46, comprising a sequence encoding a self-cleaving peptide located between a sequence encoding the CAR and a sequence encoding the Bcl-2 protein.

48. The nucleic acid molecule according to claim 47, wherein the self-cleaving peptide comprises a 2A peptide.

49-54. (canceled)

55. A composition, comprising the immune effector cell according to claim 1.

56. A method for treating diseases and/or disorders, comprising:
administering to a subject in need thereof the immune effector cell according to claim 1,
wherein the diseases and/or disorders comprise tumors.

57-66. (canceled)


SEQUENCE LISTING (62 sequences)

SEQ ID NO: 1 - protein, 13 aa, Artificial Sequence
  scFv20 HCDR3
RSTYYGGDWYFNV

SEQ ID NO: 2 - protein, 14 aa, Artificial Sequence
  scFv20 HCDR2
WIGAIYPGNGDTSY

SEQ ID NO: 3 - protein, 9 aa, Artificial Sequence
  scFv20 HCDR1
YTFTSYNMH

SEQ ID NO: 4 - protein, 8 aa, Artificial Sequence
  scFv20 LCDR3
QQWTSNPP

SEQ ID NO: 5 - protein, 11 aa, Artificial Sequence
  scFv20 LCDR2
PWIYATSNLAS

SEQ ID NO: 6 - protein, 7 aa, Artificial Sequence
  scFv20 LCDR1
SSVSYIH

SEQ ID NO: 7 - protein, 121 aa, Artificial Sequence [sequence omitted]

SEQ ID NO: 8 - protein, 106 aa, Artificial Sequence [sequence omitted]

SEQ ID NO: 9 - protein, 15 aa, Artificial Sequence
  linker
GGGGSGGGGSGGGGS

SEQ ID NO: 10 - protein, 242 aa, Artificial Sequence
  scFv20
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 11 - protein, 10 aa, Artificial Sequence
  scFvAb10 HCDR3
RVYYGNSFAY

SEQ ID NO: 12 - protein, 14 aa, Artificial Sequence
  scFvAb10 HCDR2
WIGLINPGSGGTNY

SEQ ID NO: 13 - protein, 9 aa, Artificial Sequence
  scFvAb10 HCDR1
YAFSNYLIE

SEQ ID NO: 14 - protein, 8 aa, Artificial Sequence
  scFvAb10 LCDR3
QNDYFYPF

SEQ ID NO: 15 - protein, 11 aa, Artificial Sequence
  scFvAb10 LCDR2
LLIYWASTRES

SEQ ID NO: 16 - protein, 14 aa, Artificial Sequence
  scFvAb10 LCDR1
QSLLNSGNQKNYLT

SEQ ID NO: 17 - protein, 118 aa, Artificial Sequence
  scFvAb10 VH
QVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSS

SEQ ID NO: 18 - protein, 116 aa, Artificial Sequence
  scFvAb10 VL
DIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTV

SEQ ID NO: 19 - protein, 249 aa, Artificial Sequence
  scFvAb10
DIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSS

SEQ ID NO: 20 - protein, 8 aa, Artificial Sequence
  scFvGC33 HCDR3
TRFYSYTY

SEQ ID NO: 21 - protein, 14 aa, Artificial Sequence
  scFvGC33 HCDR2
WMGALDPKTGDTAY

SEQ ID NO: 22 - protein, 9 aa, Artificial Sequence
  scFvGC33 HCDR1
YTFTDYEMH

SEQ ID NO: 23 - protein, 8 aa, Artificial Sequence
  scFvGC33 LCDR3
SQNTHVPP

SEQ ID NO: 24 - protein, 11 aa, Artificial Sequence
  scFvGC33 LCDR2
LLIYKVSNRFS

SEQ ID NO: 25 - protein, 13 aa, Artificial Sequence
  scFvGC33 LCDR1
QSLVHSNANTYLH

SEQ ID NO: 26 - protein, 115 aa, Artificial Sequence
  scFvGC33 VH
QVQLVESGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 27 - protein, 112 aa, Artificial Sequence
  scFvGC33 VL
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIK

SEQ ID NO: 28 - protein, 242 aa, Artificial Sequence
  scFvGC33
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVESGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 29 - protein, 9 aa, Artificial Sequence
  scFvGC179 HCDR3
REVTTSFAY

SEQ ID NO: 30 - protein, 17 aa, Artificial Sequence
  scFvGC179 HCDR2
WVARIRSESNNYATYYG

SEQ ID NO: 31 - protein, 9 aa, Artificial Sequence
  scFvGC179 HCDR1
FSFNINAMN

SEQ ID NO: 32 - protein, 8 aa, Artificial Sequence
  scFvGC179 LCDR3
MQHIEYPF

SEQ ID NO: 33 - protein, 11 aa, Artificial Sequence
  scFvGC179 LCDR2
LLIYWMSNLAS

SEQ ID NO: 34 - protein, 13 aa, Artificial Sequence
  scFvGC179 LCDR1
KSLLHSNGNTYLN

SEQ ID NO: 35 - protein, 119 aa, Artificial Sequence
  scFvGC179 VH
EVQLVETGGGLVQPEGSLKLSCAASGFSFNINAMNWVRQAPGKGLEWVARIRSESNNYATYYGDSVKDRFTISRDDSQNMLYLQMNNLKTEDTAIYYCVREVTTSFAYWGQGTLVTVSA

SEQ ID NO: 36 - protein, 112 aa, Artificial Sequence
  scFvGC179 VL
DIVMTQSAPSVPVTPGESVSISCKSSKSLLHSNGNTYLNWFLQRPGQSPQLLIYWMSNLASGVPDRFSGSGSGTAFTLRISRVEAEDVGVYYCMQHIEYPFTFGTGTKLEIK

SEQ ID NO: 37 - protein, 246 aa, Artificial Sequence
  scFvGC179
DIVMTQSAPSVPVTPGESVSISCKSSKSLLHSNGNTYLNWFLQRPGQSPQLLIYWMSNLASGVPDRFSGSGSGTAFTLRISRVEAEDVGVYYCMQHIEYPFTFGTGTKLEIKGGGGSGGGGSGGGGSEVQLVETGGGLVQPEGSLKLSCAASGFSFNINAMNWVRQAPGKGLEWVARIRSESNNYATYYGDSVKDRFTISRDDSQNMLYLQMNNLKTEDTAIYYCVREVTTSFAYWGQGTLVTVSA

SEQ ID NO: 38 - protein, 7 aa, Artificial Sequence
  scFvM3C11 HCDR3
VRQGGAY

SEQ ID NO: 39 - protein, 15 aa, Artificial Sequence
  scFvM3C11 HCDR2
WVAAIDSSGGDTYYL

SEQ ID NO: 40 - protein, 9 aa, Artificial Sequence
  scFvM3C11 HCDR1
FTFSRYAMS

SEQ ID NO: 41 - protein, 8 aa, Artificial Sequence
  scFvM3C11 LCDR3
WQGTHFPL

SEQ ID NO: 42 - protein, 11 aa, Artificial Sequence
  scFvM3C11 LCDR2
RLIYLVSKLDS

SEQ ID NO: 43 - protein, 13 aa, Artificial Sequence
  scFvM3C11 LCDR1
QSLLDSDGKTYLN

SEQ ID NO: 44 - protein, 114 aa, Artificial Sequence
  scFvM3C11 VH
EVQLVESGGGLVKPGGSLKLSCAASGFTFSRYAMSWVRQIPEKILEWVAAIDSSGGDTYYLDTVKDRFTISRDNANNTLHLQMRSLRSEDTALYYCVRQGGAYWGQGTLVTVSA

SEQ ID NO: 45 - protein, 112 aa, Artificial Sequence
  scFvM3C11 VL
DVVMTQTPLTLSVTIGQPASISCKSSQSLLDSDGKTYLNWLLQRPGQSPKRLIYLVSKLDSGAPDRFTGSGSGTDFTLKISRVEAEDLGIYYCWQGTHFPLTFGAGTKLELK

SEQ ID NO: 46 - protein, 241 aa, Artificial Sequence
  scFvM3C11
DVVMTQTPLTLSVTIGQPASISCKSSQSLLDSDGKTYLNWLLQRPGQSPKRLIYLVSKLDSGAPDRFTGSGSGTDFTLKISRVEAEDLGIYYCWQGTHFPLTFGAGTKLELKGGGGSGGGGSGGGGSEVQLVESGGGLVKPGGSLKLSCAASGFTFSRYAMSWVRQIPEKILEWVAAIDSSGGDTYYLDTVKDRFTISRDNANNTLHLQMRSLRSEDTALYYCVRQGGAYWGQGTLVTVSA

SEQ ID NO: 47 - protein, 24 aa, Artificial Sequence
  transmembrane domain
IYIWAPLAGTCGVLLLSLVITLYC

SEQ ID NO: 48 - protein, 42 aa, Artificial Sequence
  co-stimulatory domain
KRGRKKLLYIFKQPFMRPVQTTQEEDGCSCRFPEEEEGGCEL

SEQ ID NO: 49 - protein, 112 aa, Artificial Sequence
  intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 45 aa, Artificial Sequence
  hinge region
TTTPAPRPPTPAPTIASQPLSLRPEACRPAAGGAVHTRGLDFACD

SEQ ID NO: 51 - protein, 22 aa, Artificial Sequence
  2A peptide
GSGATNFSLLKQAGDVEENPGP

SEQ ID NO: 52 - protein, 239 aa, Artificial Sequence
  Bcl-2
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 53 - protein, 751 aa, Artificial Sequence
  20BBZ-Bcl-2
METDTLLLWVLLLWVPGSTGTGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 54 - protein, 490 aa, Artificial Sequence
  20BBZ
METDTLLLWVLLLWVPGSTGTGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 758 aa, Artificial Sequence
  Ab10BBZ-Bcl-2
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 56 - protein, 497 aa, Artificial Sequence
  Ab10BBZ
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 751 aa, Artificial Sequence
  GC33BBZ-Bcl-2
METDTLLLWVLLLWVPGSTGTGDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVESGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 58 - protein, 490 aa, Artificial Sequence
  GC33BBZ
METDTLLLWVLLLWVPGSTGTGDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVESGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 755 aa, Artificial Sequence
  GC179BBZ-Bcl-2
METDTLLLWVLLLWVPGSTGTGDIVMTQSAPSVPVTPGESVSISCKSSKSLLHSNGNTYLNWFLQRPGQSPQLLIYWMSNLASGVPDRFSGSGSGTAFTLRISRVEAEDVGVYYCMQHIEYPFTFGTGTKLEIKGGGGSGGGGSGGGGSEVQLVETGGGLVQPEGSLKLSCAASGFSFNINAMNWVRQAPGKGLEWVARIRSESNNYATYYGDSVKDRFTISRDDSQNMLYLQMNNLKTEDTAIYYCVREVTTSFAYWGQGTLVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 60 - protein, 494 aa, Artificial Sequence
  GC179BBZ
METDTLLLWVLLLWVPGSTGTGDIVMTQSAPSVPVTPGESVSISCKSSKSLLHSNGNTYLNWFLQRPGQSPQLLIYWMSNLASGVPDRFSGSGSGTAFTLRISRVEAEDVGVYYCMQHIEYPFTFGTGTKLEIKGGGGSGGGGSGGGGSEVQLVETGGGLVQPEGSLKLSCAASGFSFNINAMNWVRQAPGKGLEWVARIRSESNNYATYYGDSVKDRFTISRDDSQNMLYLQMNNLKTEDTAIYYCVREVTTSFAYWGQGTLVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 726 aa, Artificial Sequence
  M3C11BBZ-Bcl-2
METDTLLLWVLLLWVPGSTGTGDVVMTQTPLTLSVTIGQPASISCKSSQSLLDSDGKTYLNWLLQRPGQSPKRLIYLVSKLDSGAPDRFTGSGSGTDFTLKISRVEAEDLGIYYCWQGTHFPLTFGAGTKLELKGGGGSGGGGSGGGGSEVQLVESGGGLVKPGGSLKLSCAASGFTFSRYAMSWVRQIPEKILEWVAAIDSSGGDTYYLDTVKDRFTISRDNANNTLHLQMRSLRSEDTALYYCVRQGGAYWGQGTLVTVSAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 62 - protein, 487 aa, Artificial Sequence
  M3C11BBZ
METDTLLLWVLLLWVPGSTGTGDVVMTQTPLTLSVTIGQPASISCKSSQSLLDSDGKTYLNWLLQRPGQSPKRLIYLVSKLDSGAPDRFTGSGSGTDFTLKISRVEAEDLGIYYCWQGTHFPLTFGAGTKLELKGGGGSGGGGSGGGGSEVQLVESGGGLVKPGGSLKLSCAASGFTFSRYAMSWVRQIPEKILEWVAAIDSSGGDTYYLDTVKDRFTISRDNANNTLHLQMRSLRSEDTALYYCVRQGGAYWGQGTLVTVSAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR